(19) United States
(12) Patent Application Publication
(10) Pub. No.: US 2010/0166727 A1
(43) Pub. Date: Jul. 1, 2010

(54) S-Adenosylmethionine And Methylthioadensosine In Chemoprevention And Treatment Of Colon Polyps And Cancer

(75) Inventor: Shelly C. Lu, Los Angeles, CA (US)

Correspondence Address: HOGAN & HARTSON L.L.P., 1999 AVENUE OF THE STARS, SUITE 1400, LOS ANGELES, CA, 90067, US

(73) Assignee: UNIVERSITY OF SOUTHERN CALIFORNIA, Los Angeles, CA (US)

(21) Appl. No.: 12/594,567

(22) PCT Filed: Apr. 2, 2008
(86) PCT No.: PCT/US08/59188

§ 371 Date: Mar. 19, 2010

Related U.S. Application Data
(60) Provisional application No. 60/909,646, filed on Apr. 2, 2007.

(51) Int. Cl. A61K 38/43; A61K 31/7076; A61P 35/00; A61P 1/06

(52) U.S. Cl. 424/94.5; 514/46

(57) ABSTRACT

The present invention relates to S-adenosylmethionine (SAMe), methylthioadenosine (MTA) for the treatment of disease. More specifically, the invention relates to SAMe and MTA methods and compositions for the chemoprevention and treatment of colon polyps and cancer.

The present application claims the benefit of the filing date of U.S. Provisional Application No. 60/909,646, filed Apr. 2, 2007, the disclosure of which are incorporated herein by reference in its entirety.

Support from NIH grants DK51719 and AT1576 is acknowledged.

### FIELD OF THE INVENTION

The present invention relates to the use of S-adenosylmethionine (SAMe) and methylthioadenosine (MTA) in the chemoprevention and treatment of disease.

### BACKGROUND OF THE INVENTION

Colorectal cancer is the second most common fatal malignancy in the Western world, with about 150,000 new cases and accounting for 55,000 deaths in the United States each year (1). In the past 15-20 years, tremendous progress has been made on understanding the pathogenesis of this common cancer. Two types of colorectal carcinomas are recognized: LOH (for loss of heterozygosity) and MSI (for microsatellite instability) (2) and four signaling pathways are recognized in colorectal carcinogenesis: 1) Wnt, 2) K-ras, 3) transforming growth factor β (TGFβ), and 4) p53 (2). Mutations of key players in these pathways lead to inactivation of tumor suppressor function or activation of protooncogenes that favor growth. In addition, increased signaling through insulin-like growth factor receptor-1 (IGF-1R) and epidermal growth factor receptor (EGFR) has been shown to be important in colon cancer growth and metastasis (3,4). Likewise, elevated leptin levels was found to be a risk factor for colon cancer in men (5), and leptin has been shown to promote invasiveness of colon cancer cells (6). Leptin levels correlate with body mass index (BMI), and obesity is a well-recognized risk factor for colon cancer (5). Thus, it is apparent that multiple mechanisms are involved in the pathogenesis of colon cancer.

Epidermal growth factor (EGF), insulin-like growth factor-1 (IGF-1) and leptin are three well-known growth factors that have been implicated in colon cancer growth and invasion. Both higher levels of IGF-1 and IGF-1R (IGF-1 receptor) have been shown in colon cancer (4,7), and IGF-1R signaling plays an important role in tumor growth, angiogenesis and metastasis (4). Increased EGF receptor signaling has also been demonstrated to provide growth advantage, correlate with colon cancer progression and metastatic potential (3, 8). Likewise, elevated leptin levels was found to be a risk factor for colon cancer in men (5), and leptin has been shown to promote invasiveness of colon cancer cells (6). Leptin levels correlate with body mass index (BMI), and obesity is a well-recognized risk factor for colon cancer (5).

Methionine adenosyltransferase (MAT) is an essential cellular enzyme that catalyzes the formation of S-adenosylmethionine (SAMe) (9). SAMe is made in all cells and is required for cell survival (27). In mammals, the catalytic subunit of MAT is encoded by two genes, MAT1A and MAT2A, while a third gene MAT2 β, encodes for a regulatory subunit β that regulates MAT2A (9, 10). In hepatocytes, an increase in MAT2A and MAT2β expression results in decreased levels of SAMe and is associated with increased growth and malignant degeneration (9, 11-13).

SAMe can inhibit liver cell growth and induce apoptosis in liver cancer cells while protecting normal hepatocytes against apoptosis (13-15). In addition, SAMe induces apoptosis in liver cancer cell lines HepG2 and HuH-7 via the mitochondrial death pathway (14). The same effects were observed with methylthioadenosine (MTA), a metabolite of SAMe. These results are consistent with the chemopreventive action of SAMe and MTA in an in vivo model of chemical hepatocarcinogenesis in rats, which was accompanied by an increase of apoptotic bodies in atypical nodules and HCC foci in SAMe treated animals (16-17).

The influence of MAT2A and SAMe levels on cell growth in other cell types has not been studied. In hepatocytes, SAMe levels are high in quiescent and low in proliferating states (13). SAMe levels are dramatically reduced shortly after ⅔ partial hepatectomy, prior to the onset of DNA synthesis (12). Preventing the fall in SAMe level by exogenous SAMe administration inhibited hepatocyte DNA synthesis (18). Additionally, exogenous SAMe inhibits the growth of hepatoma cells (13), prevents development of HCC in rats treated with hepatocarcinogen (19, 17), and chronic SAMe depletion results in spontaneous HCC (20). One of the molecular mechanisms of SAMe's growth inhibitory response is inhibition of hepatocyte growth factor (HGF) (21). This involves inhibition of HGF-mediated activation of AMP kinase (AMPK) and HuR nuclear to cytoplasmic translocation in hepatocytes (22). It should be noted that AMPK activation in colon cancer cells resulted in the opposite effect, HuR cytoplasmic to nuclear translocation (23). Thus, the two cell types are quite different in terms of how AMPK and SAMe regulate growth.

### Kinetic Properties and Regulation of MAT Protein Function

MAT isoenzymes differ in kinetic and regulatory properties and sensitivities to inhibitors of MAT (9). MAT II has the lowest (˜4-10 μM), while MAT I has intermediate (23 μM-1 mM) and MAT III has highest (215 μM-7 mM) Km for methionine (9). SAMe strongly inhibits MAT II (IC50=60 μM, close to intracellular [SAMe] (24); whereas it minimally inhibits MAT I (IC50=400 μM) and stimulates MAT III (up to 8-fold at 500 μM SAMe) (25). Thus, SAMe level in cells that express only MAT II should be relatively unaffected by fluctuations in methionine availability because of negative feedback inhibition. One caveat is the β regulatory subunit, which renders MAT II more susceptible to feedback inhibition by SAMe (26).

Regulation of MAT at the mRNA Level

MAT1A is a marker of differentiated liver phenotype (27). MAT1A gene transcription is turned off in HCC (11), and its expression is decreased in patients with liver disease (28, 29). In contrast, MAT2A is induced transcriptionally in human HCC (11), and in rodents during rapid liver growth and dedifferentiation (12, 30, 31). Mechanisms for MAT2A induction include histone acetylation and increased trans-activating activities of c-Myb, Sp1, AP-1 and NFκB (32-35). Similar to MAT2A, MAT2β is also expressed in extrahepatic tissues but not in normal liver. In human liver, β subunit expression is increased in cirrhosis and HCC. Increased expression of the β subunit reduced SAMe content and stimulated DNA synthesis (36). Outside of the liver, the only cell type studied is T-lymphocytes, where MAT2A gene expression increase while MAT2β expression decrease during activation, which was thought to provide an increased supply of SAMe, the precursor to polyamine synthesis required for cell growth (37, 26).

While the type of MAT expressed by the cell can influence the steady state SAMe level (13), SAMe level can influence MAT expression in return. MAT1A expression falls while MAT2A is induced in primary cultures of hepatocytes, due to de-differentiation (38). This change is prevented by the addition of SAMe. A similar regulation occurs in human MAT2A as well. MAT2A gene expression is rapidly induced when SAMe falls (by restricting L-methionine in medium) and down-regulated when SAMe is added (33, 39). The molecular mechanisms are still unclear and whether SAMe regulates MAT2β is unknown. Table I summarizes properties of mammalian MATs.

TABLE 1
Properties of mammalian MATs - Kₘs differ depending on purity of the enzyme

| MAT | | Catalytic | Regulatory | Subunit | Kₘ | Tissue | Inhibition |
|---|---|---|---|---|---|---|---|
| isoform | Gene | subunit | subunit | structure | methionine | localization | by SAMe |
| MAT I | MAT1A | α1 | No | (α1)₄ | 23 μM-1 mM | Liver, pancreas | No |
| MAT III | MAT1A | α1 | No | (α1)₂ | 215 μM-7 mM | Liver, pancreas | No |
| MAT II | MAT2A | α2 | β | varies | 4-10 μM | Extrahepatic tissues | Yes |
| | | | | | | Fetal liver, HCC | |
| | MAT2β | | | | | Same as MAT2A | ? |

### SAMe Biosynthesis and Metabolism

SAMe is made in all cells and is required for survival (27) (FIG. 1). SAMe is synthesized as the first step in methionine (met) catabolism in a reaction catalyzed by MAT. SAMe is the link to three key metabolic pathways, polyamine synthesis, transmethylation and transsulfuration. In polyamine synthesis, SAMe is decarboxylated and the remaining propylamino moiety attached to its sulfonium ion is donated to putrescine to form spermidine and methylthioadenosine (MTA) and to spermidine to form spermine and a second molecule of MTA. In transmethylation, SAMe donates its methyl group to a large variety of acceptor molecules in reactions catalyzed by methyltransferase (MTs). S-adenosylhomocysteine (SAH) is generated as a product of transmethylation and is hydrolyzed to form homocysteine (Hcy) and adenosine through a reversible reaction catalyzed by SAH hydrolase. SAH is a Potent competitive inhibitor of methylation reactions and prompt removal of adenosine and Hcy is required to prevent accumulation of SAH. Hcy can be remethylated to form methionine via methionine synthase (MS), which requires folate and vitamin B12 and betaine homocysteine methyltransferase (BHMT), which requires betaine, a metabolite of choline. Remethylation of homocysteine via MS requires 5-methyltetrahydrofolate (5-MTHF), which is derived from 5,10-methylenetetrahydrofolate (5,10-MTHF) in a reaction catalyzed by methylenetetrahydrofolate reductase (MTHFR). 5-MTHF is then converted to tetrahydrofolate (THF) as it donates its methyl group and THF is converted to 5,10-MTHF to complete the folate cycle. Hcy can also undergo the transsulfuration pathway to form cysteine (the rate-limiting precursor for GSH) via a two-step enzymatic process catalyzed by cystathionine β-synthetase (CBS) and cystathionase, both requiring vitamin B6. All mammalian tissues express MAT and MS, whereas BHMT expression is limited to the liver and kidney. In the liver, SAMe plays a regulatory role on methionine metabolism by inhibiting MTHFR and MS and activating CBS (90). Thus, when SAMe is depleted, homocysteine is channeled to remethylation to regenerate SAMe; whereas when SAMe level is high, homocysteine is channeled to the trans-sulfuration pathway.

### MAT and SAMe in Liver Disease

Patients with chronic liver disease have impaired hepatic SAMe biosynthesis, due to diminished MAT1A expression and inactivation of the MATI/III enzyme (9). We examined the consequences of chronic hepatic SAMe depletion using the MAT1A knockout (KO) mouse model. MAT1A KO mice have markedly increased serum methionine levels, reduced hepatic SAMe and GSH levels. They are more prone to develop choline-deficient diet-induced fatty liver and spontaneous non-alcoholic steatohepatitis (NASH) (29). Using genomic/proteomic approaches, we identified up-regulation of hepatic cytochrome P450 2E1(CYP2E1) expression in the KOs, which explained the predisposition to CC14-induced liver injury (40), and down-regulation of prohibitin 1 from the time of birth to the development of NASH (41). Prohibitin 1 is the product of a nuclear gene that is targeted to the inner mitochondrial membrane, and act as a chaperone-like protein that participates in the correct folding and assembly of some of the mitochondrial respiratory chain proteins. By 18 months, majority of the KO mice develop HCC on a normal diet (40). Thus, the fall in MAT activity observed in human liver cirrhosis may contribute to the pathogenesis and progression of the disease as well as predisposition to HCC.

### SAMe Regulation of Growth

In hepatocytes, SAMe levels are high in quiescent and low in proliferating states (13). SAMe levels are dramatically reduced shortly after ⅔ partial hepatectomy, prior to the onset of DNA synthesis (12). Preventing the fall in SAMe level by exogenous SAMe administration inhibited hepatocyte DNA synthesis (18). Additionally, exogenous SAMe inhibits the growth of hepatoma cells (13), prevents development of HCC in rats treated with hepatocarcinogen (19, 17), and chronic SAMe depletion results in spontaneous HCC (20). One of the molecular mechanisms of SAMe's growth inhibitory response is inhibition of hepatocyte growth factor (HGF) (21). This involves inhibition of HGF-mediated activation of AMP kinase (AMPK) and HuR nuclear to cytoplasmic translocation in hepatocytes (22). It should be noted that AMPK activation in colon cancer cells resulted in the opposite effect, HuR cytoplasmic to nuclear translocation (23). Thus, the two cell types are quite different in terms of how AMPK and SAMe regulate growth.

Outside of the liver, the only cell type studied is lymphocytes, where SAMe levels increase during T lymphocyte activation due to increased MAT2A and decreased MAT2β expression (37, 42). Increased SAMe was thought to enhance polyamine synthesis necessary for growth (37). Thus, in hepatocytes, a fall in intracellular SAMe level allows the cell to proliferate (by releasing the inhibitory tone exerted on growth factors) but in T lymphocytes, the opposite occurs. If SAMe accumulation is blocked, lymphocyte proliferation is inhibited (59). The influence of SAMe in other cell types is unknown.

### SAMe Regulation of Apoptosis

While SAMe protected against okadaic acid-induced apoptosis in normal hepatocytes, it induced apoptosis in liver cancer cell lines HepG2 and HuH-7 via the mitochondrial death pathway (14). The same effects were observed with MTA. These results are consistent with the chemopreventive action of SAMe and MTA in an in vivo model of chemical hepatocarcinogenesis in rats, which was accompanied by an increase of apoptotic bodies in atypical nodules and HCC foci in SAMe treated animals (16, 17).

We identified one mechanism of SAMe and MTA's differential effect on apoptosis in normal versus cancerous liver cells (15). Bcl-x is alternatively spliced to produce two distinct mRNAs and proteins, Bcl-xL and Bcl-xS. Bcl-xL is anti-apoptotic while Bcl-xS is pro-apoptotic. SAMe and MTA induced selectively Bcl-xS in HepG2 cells by increasing alternative splicing. Furthermore, inhibitors of protein phosphatase 1 (PP1) blocked SAMe and MTA's ability to induce Bcl-xS. PP1 modulates alternative splicing by dephosphorylating SR proteins. SAMe and MTA increased PP1 catalytic subunit mRNA and protein levels and de-phosphorylated SR proteins. The effects of SAMe and MTA on Bcl-xS and apoptosis were not seen in primary hepatocytes. We found SAMe and MTA also induce apoptosis in colon cancer cells but not in NCM460 cell line, an immortalized cell line derived from normal human colonocytes (43). This differential effect of SAMe is consistent with its in vivo safety profile. SAMe is widely used in this country as a health supplement and is considered free of any significant side effects (44).

### MAT Expression in Colon Cancer

Despite the importance of MAT and SAMe in cell survival and function, regulation of MAT2A and MAT2β and role of SAMe outside of the liver and lymphocytes are essentially unknown. Only one paper has examined correlation between MAT expression and the stages of human colorectal carcinoma (45). In this study of 10 patients, MAT activity in the tumor region was higher than in the normal tissue and patients with more advanced stage (Dukes' stage C or D) had higher tumor to normal MAT activity ratios than those with Dukes' stage A or B. Higher MAT activity correlated with higher immunohistochemical staining for MAT II (45). Our hypothesis is that up-regulation in MAT2A and MAT2β expression facilitates colon cancer cell growth.

### SUMMARY OF THE INVENTION

In one embodiment, the invention relates to compositions of SAMe and/or MTA that induce apoptosis in cancer cells.

In another embodiment, the invention relates to compositions of SAMe and/or MTA for the chemoprovention of cancer cells.

In a related embodiment, the invention relates to the compositions of SAMe and/or MTA that treat cancer cells.

In yet another embodiment, the invention relates to the diagnosis of cancer via MAT overexpression.

In accordance with one embodiment, the invention relates to methods of using SAMe and/or MTA for the induction of apoptosis in cancer.

In accordance with another embodiment, the invention relates to methods of using SAMe and/or MTA for the chemoprovention of cancer.

In accordance with a related embodiment, the invention relates to methods of using SAMe and/or MTA for the treatment of cancer.

In one embodiment, the invention relates to the role of SAMe and/or MTA in the induction of apoptosis in cancer.

In yet another embodiment, the invention relates to methods of diagnosing cancer via MTA overexpression.

To practice methods relating to apoptosis of cancer, cancer cells are treated with SAMe and/or MTA and the cell viability is determined. If the cell viability of cells treated with SAMe and/or MTA is less than the cell viability of control cells, then SAMe and/or MTA has induced apoptosis.

To practice methods relating to the chemoprevention of cancer, the drinking water of mice that have been treated with an agent that causes preneoplastic lesions in the colon of mice is supplemented with SAMe and/or MTA. If the number of lesions that develop in the mice treated with SAMe and/or MTA is less than the number of lesions that develop in untreated mice, then SAMe and/or MTA has chemoprevented cancer.

To practice methods relating to the diagnosis of cancer, colon cancer specimens are examined for MAT2A by real-time PCR. If the mRNA levels are elevated in the colon cancer specimen as compared to normal colon specimens, then colon cancer has been diagnosed.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### DESCRIPTION OF THE FIGURES

FIG. 1. Schematic of SAMe metabolism.

FIG. 2. Steady state MAT II protein levels in colon cancer and HT-29 cells treated with IGF-1. Human colon cancer specimens and matched normal colon tissues (NL), and HT-29 cells treated with IGF-1 (100 ng/ml for 8 hrs) were processed for Western blot analysis using 40 μg protein/lane. Densitometric changes (% of NL or control) are shown below each blot.

FIG. 3. Increased MAT2A and MAT2β gene transcription in colon cancer. Nuclear run was performed in two patients where cancer (CA) and normal (NL) colon tissues.

FIG. 4. DNase I footprinting of MAT2A promoter in normal (NL) and cancerous (CA) colon. Lane G+A represents a Maxam-Gilbert sequencing reaction in the same fragment. The number above each lane is the amount of nuclear protein in micrograms. The DNase I protected region (indicated by brackets on the right) is in cancer, but not in the normal colon.

FIG. 5. MAT2A promoter methylation pattern in normal (NL) colon and cancer (CA) colon. Southern blot analysis was preformed using MAT2A probe (−571 to +60 bp). MspI is methylation insensitive while HpaII is not. There are more higher molecular weight bands in NL (arrows) as compared to CA, which is consistent with MAT2A being more hypomethylated in CA.

FIG. 6. Effects of leptin, IGF-1 and EGF on expression of MAT genes in RKO and HT-29 cells. (A) RKO cells were treated with leptin (100 ng/ml) for one to 24 hrs and MAT2A and MAT2β mRNA levels were measured using real-time PCR. (B) RKO cells were treated with leptin (100 ng/ml, 6 hrs), IGF-1, or EGF (both at 100 ng/ml, 3 hrs) and MAT2A mRNA levels were measured as in (A). In (C) HT-29 cells were treated with leptin as in (A). All results represent mean±SEM from three independent experiments done in duplicates. *p<0.05 vs. control.

FIG. 7. SAMe and MTA lower MAT2A mRNA levels (A) and prevent growth factors-induced increase in MAT2A expression (B). HT-29 cells were treated with SAMe (5 mM) or MTA (1 mM) or vehicle control for 3 to 6 hrs and MAT2A mRNA levels were determined by real-time PCR (A). To examine whether SAMe and MTA can modulate the effect of the growth factors on MAT2A expression, HT-29 cells were treated with growth factors, SAMe or MTA alone or in combination. Co-treatment of SAMe or MTA with leptin was for 6 hrs, while co-treatment for IGF-1 or EGF was for 3 hrs. All results represent mean±SEM from 3 to 4 independent experiments done in duplicates. *p<0.05 vs. control, †p<0.05 vs. respective growth factors.

FIG. 8. Effect of IGF-1, EGF, leptin, SAMe and MTA treatment on luciferase activity driven by the human MAT2A promoter. HT-29 cells were transfected transiently with the human MAT2A promoter construct—571/+60-LUC or pGL-3 enhancer vector and treated with IGF-1 (100 ng/ml, 3 hrs), EGF (100 ng/ml, 3 hrs), leptin (100 ng/ml, 6 hrs), SAMe (5 mM, 6 hrs), MTA (1 mM, 6 hrs), or vehicle control. Cells were co-transfected with Renilla luciferase for control of transfection efficiency. Results represent mean±SEM from three independent experiments performed in triplicates. Data is expressed as relative luciferase activity to that of pGL-3 enhancer vector control, which is assigned a value of 1.0. *p<0.05 vs. −571/+60-LUC construct control.

FIG. 9. Effects of SAMe, MTA, EGF, IGF-1 and leptin on cell growth. HT-29 cells were treated with various growth factors, SAMe (0.5 to 5 mM), MTA (0.5 to 1 mM) alone or in combination for 16 hrs for cell growth determination by the MTT assay as described in Methods. Results represent mean±SEM from three independent experiments done in triplicates. *p<0.05 vs. control, †p<0.05 vs. respective growth factors.

FIG. 10. MAT2A RNAi#1 causes a profound decrease in MAT2A mRNA level and increased apoptosis. RKO cells were treated with MAT2A RNAi#1 and MAT2A mRNA levels were determined up to 48 hrs after treatment (A). Apoptosis was determined by HOECHST staining after 24 hrs (B) and 48 hrs (C) of RNAi#1 treatment. Full time course study of RNAi#1 on MAT2A expression was done in triplicate in one experiment, apoptosis was determined in three separate experiments. *p<0.05 vs. scrambled.

FIG. 11. Effect of MAT2A RNAi#5 on MAT2A mRNA levels and cell growth in response to growth factors. In (A) HT-29 cells were treated with RNAi#5 or scrambled (SC) RNAi control for 48 hrs, then growth factors were added for either 3 hrs (IGF-1, EGF) or 6 hrs (leptin) at 100 ng/ml. MAT2A mRNA levels were measured by real-time PCR. In (B) HT-29 or RKO cells were treated with RNAi#5 or SC control for 48 hrs, followed by addition of growth factors or vehicle control for another 24 hrs for the MTT assay. *p<0.05 vs. control, †p<0.05 vs. RNAi#5 and respective growth factors from 3 experiments.

FIG. 12. Stability of SAMe and MTA in culture medium at 37° C. SAMe (A) and MTA (B) levels were measured in culture medium without cells with starting SAMe concentrations ranging from 1 to 5 mM and MTA concentration at 1 mM.

FIG. 13. Methionine, SAMe, and polyamine metabolism in cellular growth pathways. The production of polyamines, required for growth and differentiation, is dependent on intracellular SAMe. In non-hepatic cells, SAMe is made by MAT2A-encoded MAT II. We show here that MAT2A is induced by mitogens, including EGF, IGF-1, and leptin, which provide an increase in SAMe source for polyamine synthesis. When cells are incubated with extracellular SAMe, growth can be affected by several possible mechanisms. If SAMe can be transported into cells, it can inhibit the activity of MAT II by product inhibition. SAMe can also be spontaneously converted into MTA, which is known to be rapidly transported into cells. The resulting increased intracellular levels of MTA can inhibit the formation of polyamines by product inhibition. Plus (+) symbols indicate activation of a step; minus (−) symbols indicate inhibition.

FIG. 14. Relationship between MAT2β variant expression and SAMe levels.

FIG. 15. Organization of the 5′ end of human MAT2β variants (number is relative to V1 translational start).

FIG. 16. Influence of MAT2β expression on growth. (A) RNAi treated for 24 hours; (B) overexpression vectors for 72 hours; (C) Western blot using anti-V5 antibody for cells overexpressing V1 or V2 vectors.

FIG. 17. Reduced MAT2β expression leads to cell death. (A) Hoechst staining; (B) graphical representation of apoptosis; (C) phase contrast.

FIG. 18. The effect of SAMe and MTA on MAT2A expression in RKO (A) and HT29 cells (B).

FIG. 19. DNase I footprinting of MAT2A promoter in RKO cells treated with leptin, EGF or IGF-1 for 6 hours. Lane G+A represents a Maxam-Gilbert sequencing reaction in the same fragment. The number above each lane is the amount of nuclear protein in micrograms. The DNase I protected region (indicated by brackets on the right) after mitogen treatment. Certain regions are shared (i.e., −478 to −520) while others are different (−155 to −125, −100 to −71). Note similarity to protected regions in colon cancer.

FIG. 20. Effect of MAT2A RNAi treatment on MAT2A mRNA levels (A) and SAMe levels (B).

FIG. 21. MAT2A RNAi treatment results in cessation of growth (A), increased cell death, (B), and inhibition in DNA synthesis (C). RKO cells were transfected with RNAi at 30% confluence.

FIG. 22. MAT2A RNAi treatment results in increased apoptosis. Photomicrogaphs of RKO cells treated with scrambled RNAi or MAT2A RNAi for 24 hours stained with Hoechst stain (A) and graphical representation of apoptosis (B).

FIG. 23. MAT2A RNAi treatment led to depletion of polyamine levels in RKO cells. All 3 polyamines: putrescine (A), spermidine (B), and spermine (C) show a time dependant decrease.

FIG. 24. SAMe and MTA inhibit mitogen induced growth in HT-29 cells. HT-29 cells were treated with SAMe, MTA, mitogens (100 ng/ml) alone or together for 16 hours.

FIG. 25. SAMe and MTA are pro-apoptotic in RKO cells (left, 24 hour treatment), but not in NCM460 cells (right).

FIG. 26. MTA potentiates TNF-\dot{α} induced apoptosis in HT-29 cells. Cells were treated with MTA (1 mM), TNF-\dot{α} (14 ng/ml) or both for 24 hours and apoptosis was assessed by DNA fragmentation.

FIG. 27. SAMe and MTA treatment lowered c-FLIPL/S and activate caspase 8. RKO cells were treated with SAMe (0.25-5 mM) or MTA (0.25-2 mM) for 6 hrs and c-FLIP isoform mRNA levels were measured using real-time PCR (A) and protein levels of c-FLIP and caspase 8 by Western blots (B).

FIG. 28. MTA sensitizes RKO cells to TRAIL-induced apoptosis. RKO cells were treated with 1 mM MTA, 20 ng.ml 5-FU, or 10 ng/ml TRAIL alone or in combination for 24 hours. Apoptosis was determined by Annexin V with FACS.

FIG. 29. MTA induces cytochrome c release in HT-29 cells. Cells were treated with MTA (1 mM) for 24 hours, cytosol was obtained and processed for Western blot analysis.

FIG. 30. Effects of oral SAMe and MTA on colonic mRNA levels of MAT2A, MAT2 β, and cFLIP_{L}. Mice were fed SAMe (150 to 450 mg/kg/d) or MTA (75 or 150 mg/kg/d) in drinking water for 6 days. Colonic mucosa was scrapped off and gene expression measured by real-time PCR.

FIG. 31. Colonic histology in mice fed SAMe or MTA. H&E staining, ×100 for control and SAMe mg/kg/d, ×200 for MTA 75 mg/kg/d.

FIG. 32. Effect of AOM and MTA treatments on ACF in Min mice. Mice were treated with AOM±MTA or vehicle control for 6 weeks. (A) A typical ACF is indicated by the blue staining and increased crypt size. (B) MTA treated mice show a reduction in the development of lesions.

### DETAILED DESCRIPTION OF THE INVENTION

MAT is a critical cellular enzyme because it catalyzes the only reaction that generates SAMe. Recently, MAT2β has also been shown to be induced in human liver cancer and to provide a growth advantage (36). While much is known about the role of dysregulation of MAT genes in liver disease and cancer, very little is known about regulation of these MAT genes outside of the liver, and virtually nothing is known about the regulation or dysregulation of these genes in colon cancer. Although MAT is responsible for generation of SAMe, which may enhance growth through the polyamine pathway, SAMe treatment of normal and malignant hepatocytes actually inhibits growth (13, 22.) Whether SAMe or its metabolite MTA can influence growth in colon cancer cells has not been examined.

The present invention provides SAMe and/or MTA for the treatment and chemoprevention of disease. More specifically, the present invention provides SAMe and/or MTA for the treatment or chemoprevention of cancer and colon polyps.

As used herein the terms “cancer” and “cancerous” refer to or describe the physiological condition in mammals that is typically characterized by abnormal and uncontrolled cell division or cell growth. Examples of cancer include but are not limited to, carcinoma, lymphoma, blastoma, sarcoma, and leukemia. More specific examples of such cancers include breast, brain, bladder, prostate, colon, intestinal, squamous cell, lung, stomach, pancreatic, cervical, ovarian, skin, colorectal, endometrial, salivary gland, kidney, thyroid, various types of head and neck cancer, polyps, and the like. More preferably the term cancer relates colon cancer.

The term “chemoprevention” refers to or describes the use of any chemical, drug, or agent to treat a disease or condition and limit its further progress, or to prevent the condition from ever occurring. Examples of the diseases or conditions include but or not limited to cancer, polyps, and the like.

“Mammal” for purposes of treatment refers to any animal classified as a mammal, including humans, mice, primates, rabbits, rats, cats, dogs, and the like.

Gene expression levels may be detected and quantified at the mRNA or protein level using a number of means well known in the art. To detect mRNAs or measure mRNA levels, cells in biological samples (e.g., tissues and body fluids) may be lysed and the mRNA in the lysates or in RNA purified or semi-purified from the lysates detected or quantified by any of a variety of methods familiar to those in the art. Such methods include, without limitation, hybridization assays using detectably labeled gene-specific DNA or RNA probes and quantitative or semi-quantitative RT-PCR (e.g., real-time PCR) methodologies using appropriate gene-specific oligonucleotide primers. Alternatively, quantitative or semi-quantitative in situ hybridization assays can be carried out using, for example, unlysed tissues or cell suspensions, and detectably (e.g., fluorescently or enzyme-) labeled DNA or RNA probes. Additional methods for quantifying mRNA levels include RNA protection assay (RPA), cDNA and oligonucleotide microarrays, and colorimetric probe based assays.

Methods for detecting proteins or measuring protein levels in biological samples are also known in the art. Many such methods employ antibodies (e.g., monoclonal or polyclonal antibodies) that bind specifically to target proteins. In such assays, an antibody itself or a secondary antibody that binds to it can be detectably labeled. Alternatively, the antibody can be conjugated with biotin, and detectably labeled avidin (a polypeptide that binds to biotin) can be used to detect the presence of the biotinylated antibody. Combinations of these approaches (including “multi-layer sandwich” assays) familiar to those in the art can be used to enhance the sensitivity of the methodologies. Some of these protein measuring assays (e.g., ELISA or Western blot) can be applied to body fluids or to lysates of test cells, and others (e.g., immunohistological methods or fluorescence flow cytometry) applied to unlysed tissues or cell suspensions. Methods of measuring the amount of a label depend on the nature of the label and are known in the art. Appropriate labels include, without limitation, radionuclides (e.g., 125I, 131I, 35S, 3H, or 32P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Other applicable assays include quantitative immunoprecipitation or complement fixation assays.

Therapeutic agents are formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

In one embodiment, the therapeutic agents are prepared with carriers that will protect the compounds against rapid elimination from the body, such as a controlled release formulation, including implants and microencapsulated delivery systems. Methods for preparation of such formulations will be apparent to those skilled in the art.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of an active compound calculated to produce the desired therapeutic effect in association with the required pharmaceutical carrier.

The dosage required for treating a subject depends on the choice of the route of administration, the nature of the formulation, the nature of the subject's illness, the subject's size, weight, surface area, age, and sex, other drugs being administered, and the judgment of the attending physician. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Materials and Methods

### Materials

Cell culture media, fetal bovine serum, and primers were obtained from Gibco BRL Life Technologies (Grand Island, N.Y.). The Luciferase Assay System was obtained from Promega (Madison, Wis.). All restriction endonucleases were obtained from either Promega or Gibco. RNAi against MAT2A was obtained from Invitrogen (Carlsbad, Calif.). SAMe in the form of disulfate p-toluenesulfonate spray dried powder (97.18% purity) was generously provided by Gnosis SRL (Cairate, Italy). MTA was purchased from Sigma (St. Louis, Mo.). All other reagents were of analytical grade and were obtained from commercial sources.

### Source of Normal and Cancerous Colon Tissue

Colon cancer and paired normal colon specimens from 13 patients, which were from a repository based on availability of both normal and cancerous colon specimens from the same patient. Tissues were immediately frozen in liquid nitrogen for subsequent analysis of mRNA, Western blot and SAMe levels as described below.

### Methods

### Experiments in Mice

Nine adenomatous polyps were obtained from three 3-month old male Min mice (four from different parts of the small intestine from one mouse, four from different parts of the colon from two other mice). Normal intestinal tissues adjacent to the polyps were included for comparison. These tissues were immediately snap frozen for subsequent analysis of mRNA and SAMe levels.

Three-month old male C57/B6 mice were fed ad libitum a standard diet (Harland Tekiad irradiated mouse diet 7912, Madison, Wis.), caged individually and had free access to water supplemented with SAMe (75 or 150 mg/kg/d), MTA (75 mg/kg/d), phosphate buffered saline (vehicle for SAMe) or DMSO (0.2%, vehicle for MTA) for 6 days. SAMe and MTA were made fresh daily. The amount of water intake, body weight, and animal behavior were closely monitored. After 6 days, animals were sacrificed, intestine was cut open along the longitudinal axis, and mucosa was stripped from the intestine (46). Mucosa was weighed and a portion processed for SAMe measurement as described below, the rest snap frozen for subsequent RNA extraction.

Cell Culture and Treatment with Growth Factors, SAMe and MTA

HT-29 and RKO cells were obtained from the Cell Culture Core of the USC Liver Disease Research Center and grown according to instructions provided by the American Type Culture Collection (ATCC, Rockville, Md.). Prior to treatment with growth factors, SAMe or MTA, medium was changed to 0.1% FBS overnight. Medium was then changed to withhold serum and cells were treated with leptin, IGF-1 or EGF (all at 100 ng/ml), SAMe (0.5 mM to 5 mM), MTA (0.5 to 1 mM), or respective vehicle controls for one to 24 hr for various assays as described below. These growth factors were shown to exert mitogenic effects in colon cancer cell lines at the dose chosen (4, 6, 47).

### RNA Interference

RNA interference (RNAi) experiments were performed using Lipofectamine RNAiMax (Invitrogen) according to the manufacturer's instructions. Small interfering RNA (siRNA) oligonucleotides for MAT2A and scrambled siRNA were synthesized by the USC Norris Comprehensive Cancer Center Microchemical Core Laboratory, and annealed to form duplexes. Stealth RNAi for MAT2A and Stealth RNAi negative control duplexes were synthesized by Invitrogen (Carlsbad, Calif.). The following siRNA sequences were used: si-MAT2A #1, 5′-ACACAUUGGAUAUGAUGAUTT-3′ (sense) and 5′-AUCAUCAUAUCCAAUGUGUTT-3′ (antisense); si-control with scrambled sequence (negative control siRNA having no perfect matches to known human genes), 5′-UUCUCCGAACGUGUCACAUdTdT-3′ (sense) and 5% AUGUGACACGUUCGGAGAAdTdT-3′ (antisense); stealth RNAi-MAT2A #5, 5′-CCACCUACAGCCAAGUGGCAGAUUU-3′ (sense) and 5′-AAAUCUGCCACUUGGCUGUAGGUGG-3′ (antisense). Transfection was allowed to proceed 72 hours before collection for different assays.

### RNA Isolation and Gene Expression Analysis

Total RNA was isolated by the EZgeno total RNA isolation kit (Genemega, San Giego, Calif.), and subjected to reverse transcription (RT) by using M-MLV Reverse transcriptase (Invitrogen). 2 μl of RT product was subjected to quantitative real-time PCR analysis. The primers and TaqMan probes for MAT2A, MAT2β and Universal PCR Master Mix were purchased from ABI (Foster City, Calif.). Hypoxanthine phosphoribosyl-transferase 1 (HPRT1) and ubiquitin C (UBC) were used as housekeeping genes (48). The thermal profile consisted of 1 cycle at 95° C. for 15 minutes followed by 40 cycles at 95° C. for 15 seconds and at 60° C. for 1 minute. The expression of MAT2A and MAT2β was checked by normalizing the Ct of MAT2A and MAT2β to that of the control housekeeping gene (HPRT1 or UBC) (49). The delta Ct obtained was used to find the relative expression of MAT genes according to the formula: Relative expression=2·^{Δ}Ct, where ΔCt=ΔCt of MAT genes in colon cancer or treated cells—ΔCt of MAT genes in normal colon or control cells.

### Human MAT2A and Glutathione Synthetase (GSS) Promoter Constructs

The human MAT2A promoter construct±571/+60-LUC and GSS promoter construct −1686/−46-LUC were previously described (33, 34, 50), and subcloned in the sense orientation upstream of the luciferase coding sequence of the pGL-3 enhancer vector (Promega). Both promoter constructs contain maximal promoter activity.

### Effect of Mitogens on MAT2A Puromoter Activity in HT-29 Cells

To study the effect of mitogens on human MAT2A promoter activity in HT-29 cells, HT-29 cells (5×10⁵ cells in 2 ml serum-free medium) were transiently transfected with 2 μg MAT2A promoter firefly luciferase gene construct or promoterless pGL3-enhancer vector (as negative control) using the Superfect Transfection Reagent (Qiagen, Valencia, Calif.) (34). To control for transfection efficiency, cells were co-transfected with the Renilla phRL-TK vector (Promega, Madison, Wis.). Cells were treated with mitogens (100 ng/ml), SAMe (5 mM), MTA (1 mM), or vehicle control during the last three (IGF-1 and EGF) to six hrs (leptin, SAMe and MTA) of the transfection (18 hrs total). The luciferase activity driven by the MAT2A promoter construct was normalized to Renilla luciferase activity. Each experiment was done with triplicate samples.

Specificity of SAMe and MTA on promoter activity was examined by transfecting HT-29 cells with the GSS promoter construct and treating cells with SAMe or MTA as above.

### Measurement of Cell Growth

Cell growth was measured by the cell growth determination kit MTT from Sigma (St. Louis, Mo.). The MTT assay measures the cell proliferation rate and reduction in cell viability. HT-29 or RKO cells (1×10⁴/well) were plated in 96-well plates and treated with growth factors (all 100 ng/ml), SAMe (0.5 to 5 mM), MTA (0.5 to 1 mM), alone or in combination for 16 hrs in serum-free medium. To determine the effect of MAT2A RNAi on mitogen-induced changes in MTT, cells were first treated with RNAi for 48 hrs followed by mitogen treatment for another 24 hrs.

### Measurement of Apoptosis

Apoptosis was assessed by HOECHST staining (34). Briefly, RKO cells were grown on coverslips and treated with RNAi#1 for 24 hrs and cells were fixed with paraformaldehyde and stained with 8 μg/ml HOECHST 33258 dye for 30 min. Cells with bright, fragmented, condensed nuclei were identified as apoptotic cells using the Nikon Eclipse TE300 fluorescent microscope. At least 5 random fields (at ×300) were counted.

### SAMe, MTA and Polyamine Levels

Cellular SAMe and MTA levels were measured (51) and polyamine levels were determined (52).

### Western Blot Analysis

Western blot analysis for MAT II in colon cancer and HT-29 cells treated with IGF-1 (100 ng/ml) for 8 hrs using anti-MAT II antibodies (GenWay Biotech, Inc., San Diego, Calif.) (15).

### Statistical Analysis

Data are given as mean±SEM. Statistical analysis was performed using Student's t-test for comparison of paired samples and ANOVA followed by Fisher's test for multiple comparisons. Significance was defined by p<0.05.

### Results

### MAT2A Expression is Induced in Human Colon Cancer

MAT expression was examined in colon cancer and adjacent normal tissue control by real-time PCR. Colon cancer and paired normal colon specimens from 13 patients, which were randomly selected from a repository based on availability of both normal and cancerous colon specimens from the same patient were available for use. Table II summarizes the clinical and molecular data. Note that MAT2A is induced in 12 of 13 colon cancer specimens. By comparison, the gene that encodes the regulatory subunit MAT2β is not significantly changed as compared to normal colon. MAT1A is not expressed in normal or cancerous colon. Increased MAT II (α2 and α2′) level was confirmed using Western blot analysis (FIG. 2). The smaller β2′ subunit is believed to be derived from α2 by post-translational modification (26). Consistent with this, SAMe levels were higher in all colon cancer specimens (normal colon=0.26±0.03, colon cancer=0.73±0.11 nmol/mg protein, results are mean±SE from six specimens each, p<0.01 by paired Student's t-test). Tissue MTA level was below detection limit.

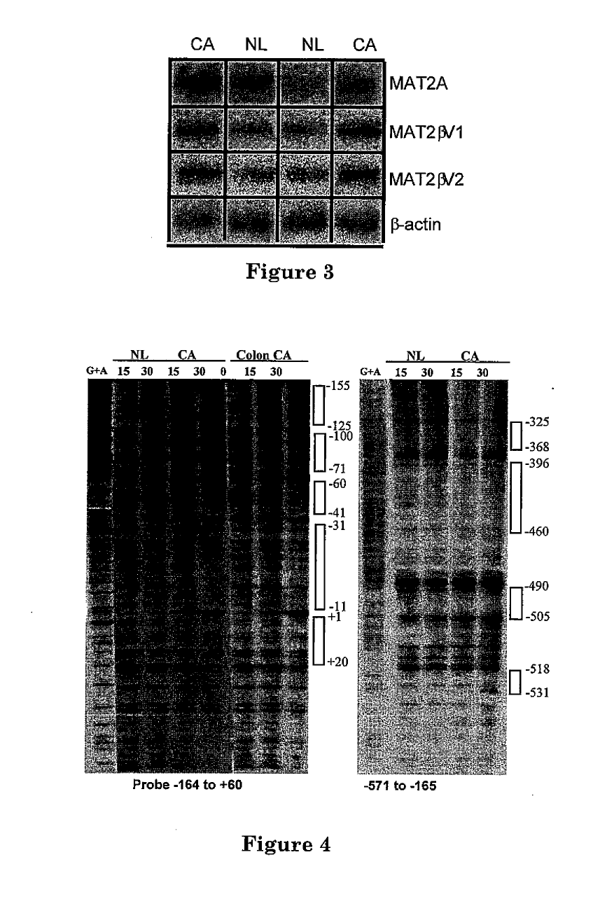

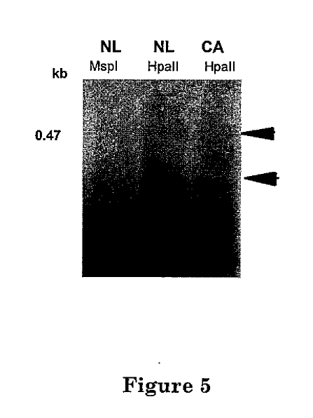

MAT2A mRNA levels were elevated in 12 of 13 cancer specimens as compared to normal tissues from the same patients (209±15%, expressed as mean±SE). Steady state SAMe levels were increased in colon cancer specimens (normal colon=0.26±0.03, colon cancer=0.73±0.11 nmol/mg protein, results are expressed as mean*SE from six matched specimens, p<0.01). Interestingly, we have uncovered two variants of MAT2β (see below) and that while variant 1 (V1) is doubled in 4 of 13 colon cancer specimens, variant 2 (V2) is induced in 9 of 13 cancer specimens to higher levels (3.0±0.4 fold of normal colon). MAT1A is not expressed in normal or cancerous colon. To see if the increase in mRNA levels of MAT2A and MAT2β in select patients is due to increased gene transcription, nuclear run on was performed. FIG. 3 shows that in two patients where the mRNA levels of MAT2A, MAT213V1 and V2 are increased by 2 to 3-fold, the mechanism is due to increased transcription. Differences in transcription can occur due to difference in transcription factor(s) binding to key cis-acting elements, which can be facilitated by a change in chromatin structure such as DNA hypomethylation. To test these hypotheses, DNase I footprinting was performed (FIG. 4) and it clearly shows that there is increased protein binding to the MAT2A promoter region in colon cancer as compared to normal colon tissue. The DNase protected regions include many Sp1, AP-1 GATA-1, E2F consensus elements. Interestingly, this footprinting pattern in colon cancer differs from HCC (32), which suggests different molecular mechanism(s) are likely involved in increased MAT2A expression in these two cancers. Using Southern blot analysis and restriction enzymes that are either sensitive (HpaII) or insensitive (MspI) to methylation status, MAT2A is relatively hypomethylated in the promoter region in colon CA (FIG. 5).

TABLE II
MAT expression in normal colon and colon cancer
specimens

| Patient # | Gender | Age | Cancer site | Stage | Histology | MAT2A* | MAT2β* |
|---|---|---|---|---|---|---|---|
| 1 | M | 65 | Rectosigmoid | T3N1M0 | Moderate | 222 | 31 |
| 2 | F | 47 | Rectum | TXN2M0 | Unknown | 171 | 90 |
| 3 | M | 53 | Rectosigmoid | T3N1M0 | Poor | 152 | 189 |
| 4 | M | 66 | Rectum | T3N1M1 | Moderate | 174 | 220 |
| 5 | M | 59 | Sigmoid | T3N2M1 | Moderate | 53 | 114 |
| 6 | F | 61 | Left colon | T3N2M1 | Moderate | 203 | 122 |
| 7 | F | 77 | Right colon | T2N2M1 | Well-diff | 245 | 88 |
| 8 | M | 67 | Right colon | T3N2M1 | Unknown | 138 | 174 |
| 9 | M | 62 | Rectum | T3N2M0 | Moderate | 171 | 103 |
| 10 | F | 56 | Right colon | Unknown | Moderate | 187 | 60 |
| 11 | M | 52 | Rectum | T3N2M0 | Poor | 295 | 153 |
| 12 | M | 73 | Rectum | TXNXM1 | Moderate | 266 | 89 |
| 13 | F | 68 | Right colon | T4N1M1 | Poor | 284 | 234 |

*Percentage relative expression, tumor:normal colon. MAT expression determined by real-time PCR.

MAT2A Expression is also Induced in Polyps of Min Mice

MAT2A mRNA levels in nine adenomatous polyps were compared to adjacent normal tissues from three Min mice and all of the polyps have higher MAT2A mRNA levels (338±93%, range 132% to 909%, expressed as percentage of matched normal tissue mRNA level, p<0.05). SAMe levels in four polyps (average MAT2A mRNA levels were 200±50% of normal) were also higher than adjacent normal tissues (normal=0.28±0.38, polyps=0.38±0.22 nmol/mg protein p<0.05). MTA levels were unchanged (normal 0.06±0.03, polyps=0.08±0.04 nmol/mg protein).

### Leptin, IGF-1 and EGF Induce MAT2A but not MAT2β Expression in RKO and HT-29 Cells

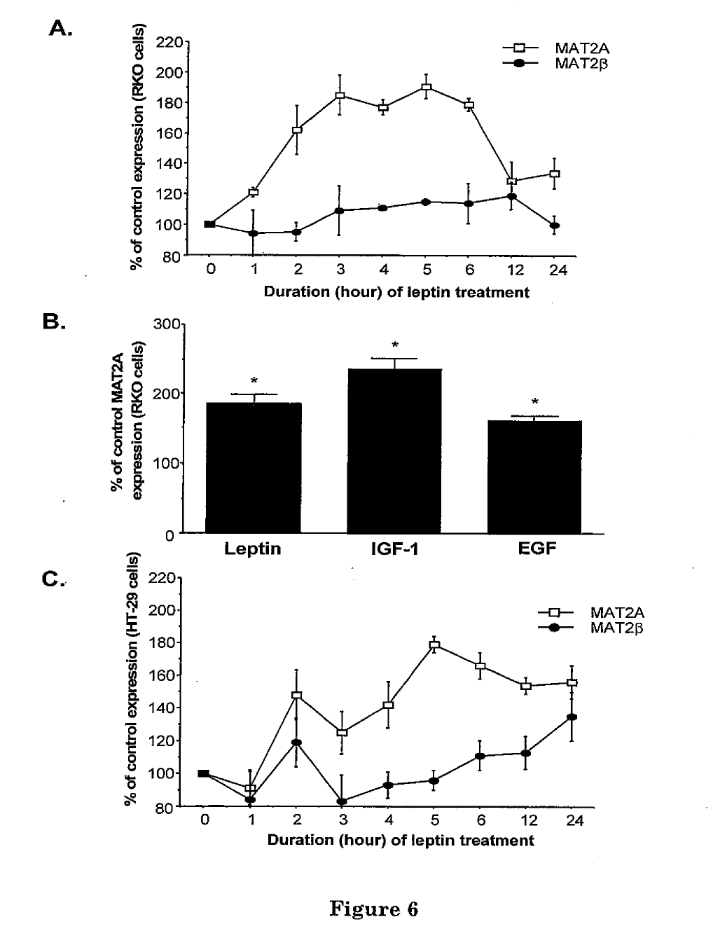

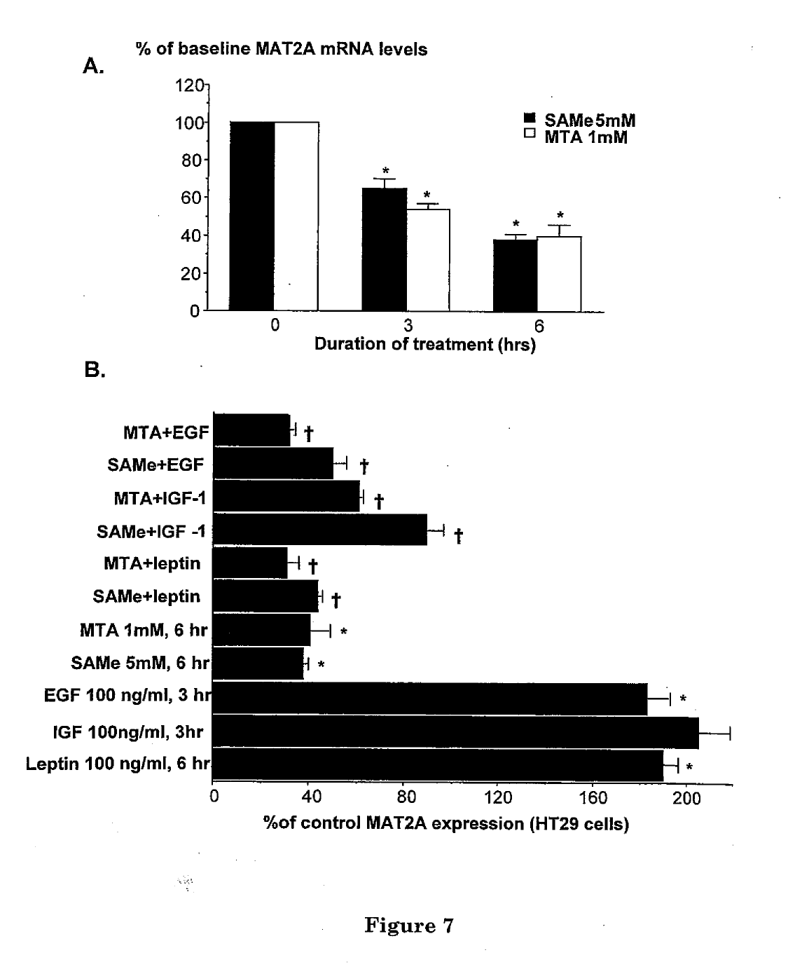

The effect of leptin on these two MAT genes in RKO and HT-29 cells were determined. The results indicated that leptin treatment resulted in a time-dependent increase in MAT2A but not MAT2β mRNA levels, with near doubling of the MAT2A mRNA levels 5 hrs after leptin treatment in both cell types (FIGS. 6A and 6C). IGF-1 and EGF treatment of both cell types also doubled the MAT2A mRNA levels (FIG. 6B and FIG. 7B). Similar to leptin, these growth factors also did not affect MAT2β mRNA level in the two colon cancer cell lines. IGF-1 treatment also increased MAT II level in HT-29 cells (FIG. 2).

### SAMe and MTA Lower Baseline MAT2A Expression and Prevent Mitogen-mediated Induction of MAT2A Expression

It was previously shown that SAMe treatment lowers MAT2A expression in hepatocytes (38, 32). Similar to hepatocytes, SAMe also lowers basal MAT2A expression in HT-29 cells in a time-dependent fashion. With 6 hr treatment, SAMe lowers MAT2A expression in a dose-dependent manner (0.5 mM SAMe=81% baseline, 1 mM SAMe=65% baseline, 2 mM SAMe=55% baseline, 5 mM SAMe=38% of baseline). MTA, an important metabolite of SAMe, also lowers basal MAT2A expression to comparable levels even at a lower dose (FIG. 7A). Next we determined whether SAMe or MTA can modulate the effect of mitogens on MAT2A expression. FIG. 7 shows that both agents are able to prevent (leptin and EGF) or significantly blunt (IGF-1) the mitogen-induced MAT2A expression. MTA is more efficient than SAMe in blocking the mitogen's effect on MAT2A expression at ⅕th the dose.

### Effects of Mitogens, SAMe and MTA on the Human MAT2A Promoter in HT-29 Cells

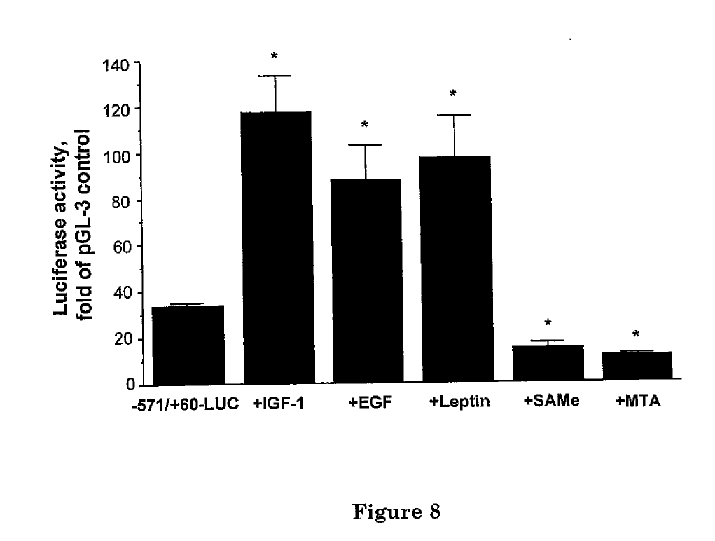

To test whether the effect of the mitogens, SAMe and MTA on MAT2A lies at the transcriptional level, their effects on the activity of the human MAT2A promoter. FIG. 8 shows that these mitogens more than double the human MAT2A promoter activity and both SAMe and MTA lower the promoter activity to comparable levels as their effects on the endogenous MAT2A expression. Importantly, SAMe and MTA treatment had no influence on the human GSS promoter activity.

### SAMe and MTA can Block the Mitogenic Effect of Leptin, EGF and IGF-1 in Colon Cancer Cells

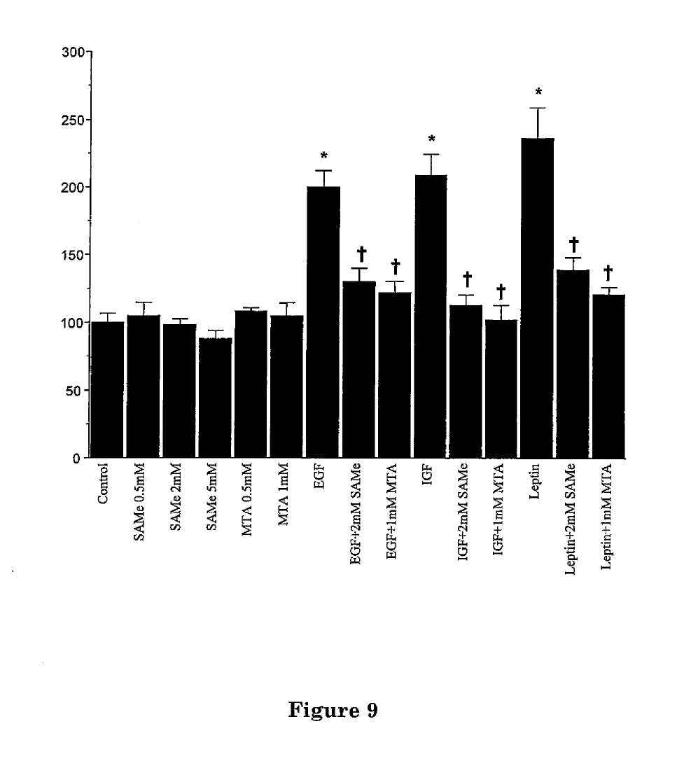

To determine whether SAMe and MTA can modulate the mitogenic effects of these growth factors. All three growth factors increased growth of HT-29 cells (similar effects were seen with RKO cells). Interestingly, while SAMe (0.5 to 5 mM) and MTA (0.5 to 1 mM) by themselves had no effect on growth after 16 hrs of treatment, they blocked the mitogenic action of all three growth factors (FIG. 9). High dose SAMe (5 mM) treatment for longer duration (24 hr) inhibited growth (71% of control).

MAT2A Expression is Closely Correlated with Growth and Cell Death

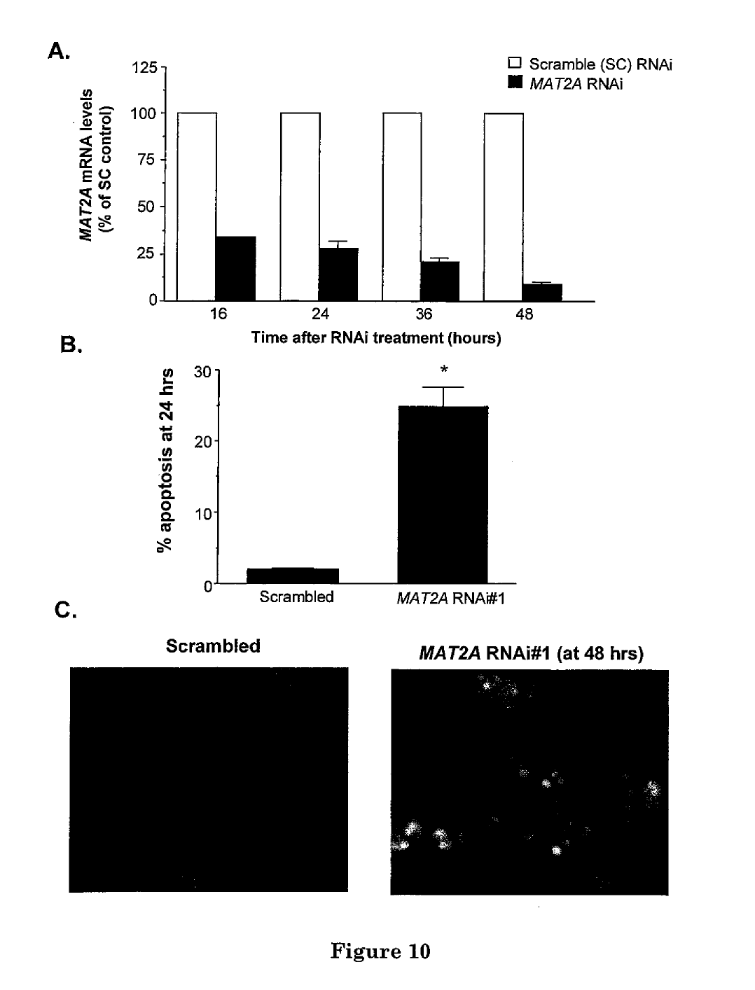

In hepatocytes, MAT2A expression correlates with growth (9, 12, 13). To determine if the increase in MAT2A expression induced by mitogens can be responsible for the increase in growth in colon cancer cells, the inventor lowered the MAT2A expression using RNAi. Several RNAi constructs were examined. The most efficient RNAi (RNAi#1) lowered MAT2A expression to 9% of scrambled control by 48 hrs (FIG. 10A). Even by 24 hrs where the expression was down to 25% of scrambled control, a significant increase in apoptosis was detected (FIG. 1013) and became more pronounced at 48 hrs (FIG. 10C).

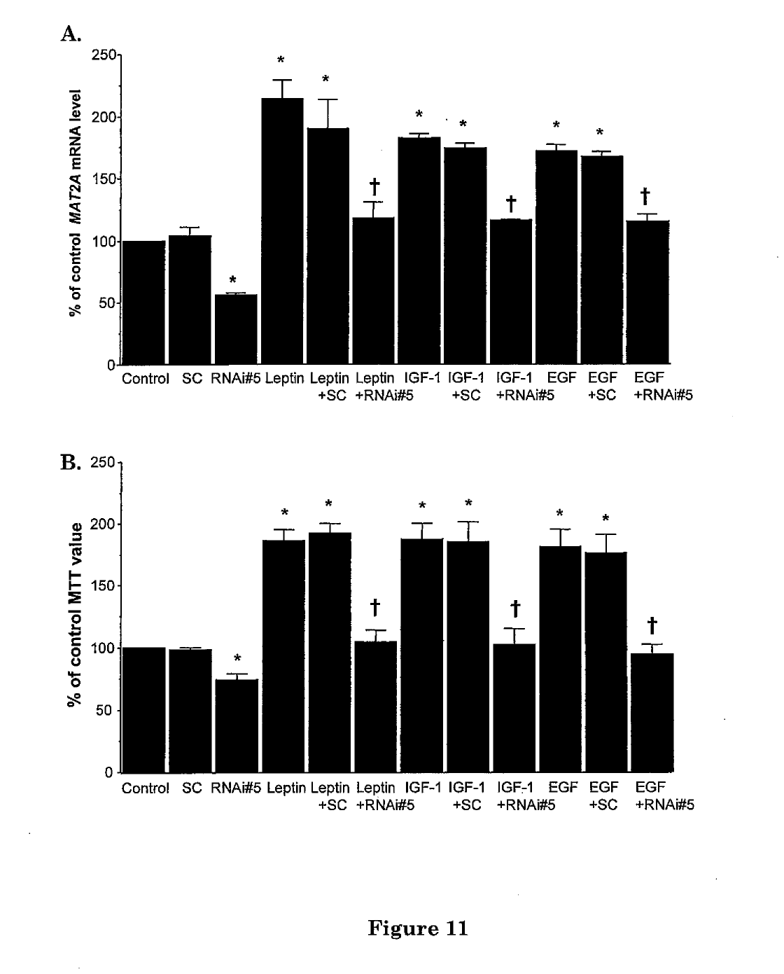

Since RNAi#1 caused cell death, a different RNAi construct that would only lower the MAT2A expression by 50% at 72 hrs (RNAi#5) was used. No apoptosis was detected with this construct after 24 hrs. This construct was used to blunt the ability of the mitogens to induce MAT2A expression. FIG. 11A shows that RNAi#5 lowered MAT2A mRNA level to 50% of control and mitogens were able to raise MAT2A mRNA levels in the presence of RNAi#5 but only back to untreated or scrambled RNAi levels. FIG. 11B shows the effect of RNAi#5 on growth. RNAi#5 lowered the MTT value by about 30%, and blocked the ability of leptin, IGF-1 and EGF to fully exert their mitogenic effect. All three growth factors were able to increase the MTT value in RNAi#5 treated cells back to baseline.

### Effect of Mitogens, SAMe and MTA on Cellular SAMe, MTA, and Polyamine Levels

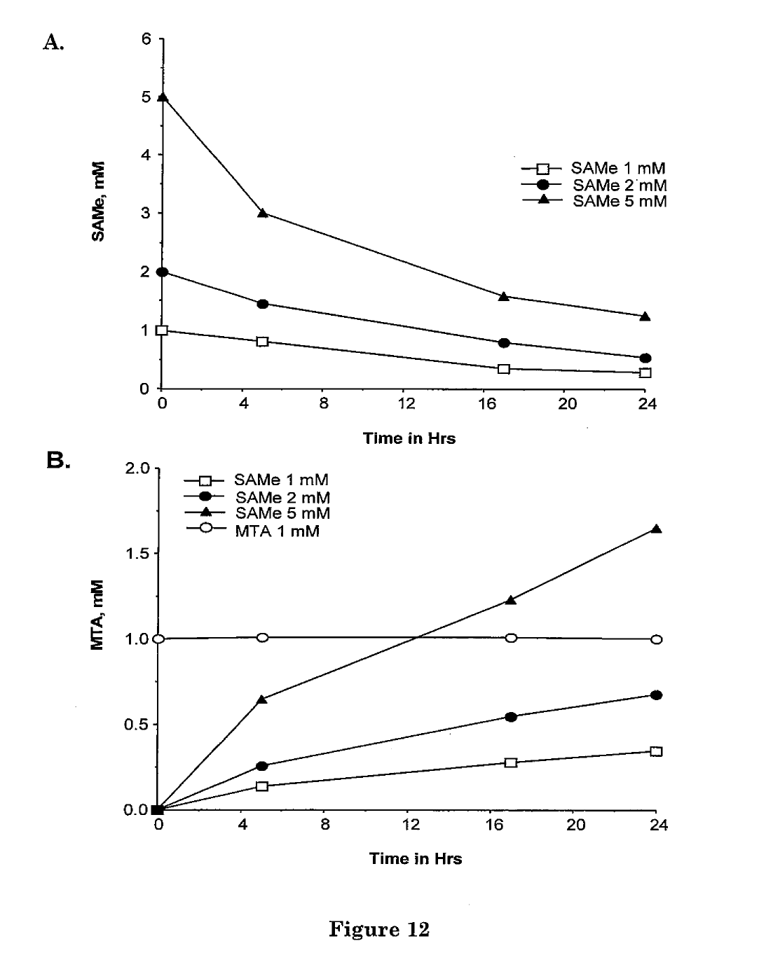

To test whether the mitogens-induce increase in. MAT2A is to provide increased SAMe necessary for polyamine synthesis in growing cells, SAMe and polyamine levels were measured following treatment of HT-29 cells with mitogens. Table III shows that all mitogens increased intracellular SAMe levels by 16 hrs of treatment. However, SAMe and MTA also raised SAMe levels. MTA can raise SAMe level because it is converted back to SAMe via the methionine salvage pathway (53). Intracellular MTA levels increased dramatically after SAMe treatment (1,906% with 5 mM SAMe for 16 hrs). In fact, SAMe treatment raised intracellular MTA levels by much higher magnitudes than SAMe levels itself, especially at high SAMe concentration. This can be explained by the fact that SAMe is highly unstable, can be converted to MTA spontaneously and also via the polyamine pathway (53). The half life of SAMe (between 1 to 5 mM) in culture medium without cells at 37° C. is 12 hrs (FIG. 12). On the other hand, the rate of conversion from SAMe to MTA is 0.013 mM per hour per mM SAMe under the same condition (FIG. 12, rate determined using linear regression). In contrast, MTA is highly stable (FIG. 12). SAMe is much more stable in water at room temperature with the half life of 36 hrs.

TABLE III
SAMe and MTA levels in HT-29 cells

| | | |
|---|---|---|
| Treatment | SAMe | MTA |

Duration: 6 hrs

| | | |
|---|---|---|
| Control | 0.40 ± 0.03 | 0.050 ± 0.007 |
| EGF | 0.54 ± 0.04* | 0.054 ± 0.001 |
| IGF-1 | 0.40 ± 0.03 | 0.054 ± 0.003 |
| Leptin | 0.48 ± 0.04 | 0.058 ± 0.010 |
| SAMe 2 mM | 1.44 ± 0.13* | 0.192 ± 0.036* |
| SAMe 5 mM | 2.19 ± 0.21* | 0.354 ± 0.096* |
| MTA 1 mM | 0.81 ± 0.09* | 0.652 ± 0.123* |

Duration: 16 hrs

| | | |
|---|---|---|
| Control | 0.42 ± 0.04 | 0.049 ± 0.005 |
| EGF | 0.63 ± 0.06* | 0.061 ± 0.010 |
| IGF-1 | 1.03 ± 0.03** | 0.087 ± 0.025 |
| Leptin | 0.63 ± 0.02* | 0.064 ± 0.015 |
| SAMe 2 mM | 1.28 ± 0.01** | 0.261 ± 0.035* |
| SAMe 5 mM | 1.83 ± 0.01** | 0.934 ± 0.105* |
| MTA 1 mM | 0.54 ± 0.01* | 0.689 ± 0.108* |

The unit for all metabolites is nmol/mg protein. Results represent mean ± SEM from 3 to 4 experiments.
*p < 0.05,
**p < 0.005 vs. respective controls

Table IV shows the effect of these treatments on the levels of polyamines. Treatment with mitogens for 6 hrs increased putrescine levels significantly (Table IV). However, SAMe and MTA treated groups had lower putrescine levels and the MTA treated group also had lower spermidine and spermine levels.

TABLE IV
Effect of mitogens, SAMe and MTA on polyamine levels
in HT-29 cells

| | | | | | | |
|---|---|---|---|---|---|---|
| ID | Putrescine | Spermidine | Spermine | ODC | SPD syn | SPM syn |

Treatment duration: 6 hrs

| | | | | | | |
|---|---|---|---|---|---|---|
| Control | 0.14 ± 0.02 | 2.59 ± 0.17 | 12.15 ± 1.50 | 1.0 ± 0.0 | 1.0 ± 0 | 1.0 ± 0 |
| EGF | 0.32 ± 0.01* | 2.19 ± 0.23 | 12.55 ± 2.04 | 1.5 ± 0.07* | 1.7 ± 0.08* | 1.1 ± 0.02 |
| IGF-1 | 0.22 ± 0.02* | 2.82 ± 0.09 | 12.55 ± 2.50 | 1.5 ± 0.08* | 2.2 ± 0.20* | 1.1 ± 0.03 |
| Leptin | 0.27 ± 0.02* | 2.62 ± 0.02 | 14.62 ± 2.70 | 1.6 ± 0.09* | 2.3 ± 0.25* | 1.0 ± 0.05 |
| SAMe 2 mM | 0.06 ± 0.01* | 2.37 ± 0.02 | 12.39 ± 2.86 | 1.1 ± 0.07 | 1.1 ± 0.02 | 1.1 ± 0.02 |
| SAMe 5 mM | 0.01 ± 0.01* | 1.90 ± 0.18 | 13.21 ± 2.58 | 0.8 ± 0.04* | 0.8 ± 0.02* | 1.0 ± 0.01 |

The units for all metabolites is nmol/mg protein.
ND = not detectable.
Expression of ornithine decarboxylase (ODC), spermine synthase (SPD syn), and spermine synthase (SPM syn) was determine by real-time PCR and expressed as fold of control.
Results represent mean ± SEM from 3 to 4 experiments.
*p < 0.05 vs. repective controls.

### MAT Expression and SAMe Levels in Colon Cancer Cell Lines

MAT expression and SAMe levels were next determined in 10 randomly selected colon cancer cell lines. Expression of each gene is expressed as relative to the value of pooled samples from these 10 cell lines. MAT2A and MAT2βV1 expression are relatively stable among the different cell lines, varying no more than 2-fold. However, MAT2βV2 expression is much more variable, varying up to 9-fold of average (Table V).

TABLE V
MAT expression and SAMe levels in colon cancer cell
lines

| | | | | | SAMe |
|---|---|---|---|---|---|
| | | | | | level |
| | | | | | (nmol/ |
| | | | | | mg |
| Cell Line | MAT1A | MAT2A | MAT2βV1 | MAT2βV2 | protein) |
| SW837 | (—) | 0.10 | 2.5 | 1.34 | 0.23 |
| LoVo | 1.41 | 1.24 | 1.44 | 8.93 | 0.71 |
| Colo205 | (—) | 0.99 | 1.89 | 3.92 | 0.45 |
| HT29 | (—) | 1.57 | 2.62 | 2.52 | 0.34 |
| RKO | (—) | 0.68 | 1.04 | 7.08 | 0.44 |
| Colo320 | (—) | 1.33 | 1.21 | 0.31 | |
| CaCo-2 | 6.70 | 1.13 | 2.3 | 2.18 | 0.20 |
| SW403 | (—) | 1.79 | 1.4 | 1.7 | 0.26 |
| Sw1417 | (—) | 1.40 | 2.45 | 2.09 | |
| SW948 | 0.46 | 1.22 | 1.27 | 1.56 | 0.22 |

MAT mRNA levels were determined by real-time PCR and expressed as relative to the level of pooled samples
MAT2βV1 mRNA level is 8-fold higher as compared to MAT2βV2 (pooled average).

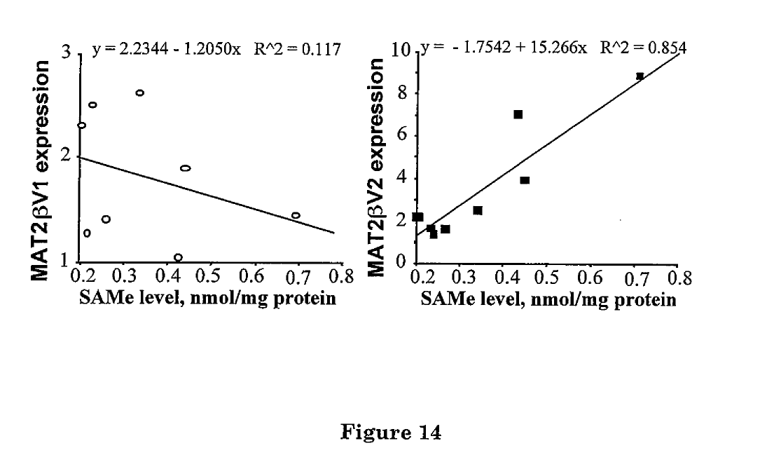

Although MAT2βV1 predominates in all cell lines, there is no correlation between its expression and SAMe level (FIG. 14 left). In contrast, there is a positive correlation between MAT2βV2 expression and SAMe level (FIG. 14 right). The β subunit is thought to regulate MAT II activity by lowering the Kₘ and Kᵢ, so a higher expression would be expected to have lower SAMe levels (26). However, it should be noted that the known function of the β subunit pertains only to variant 1 (26, 54, 36), and these data would suggest that the two β variants have distinct functions.

### MAT2β Variants

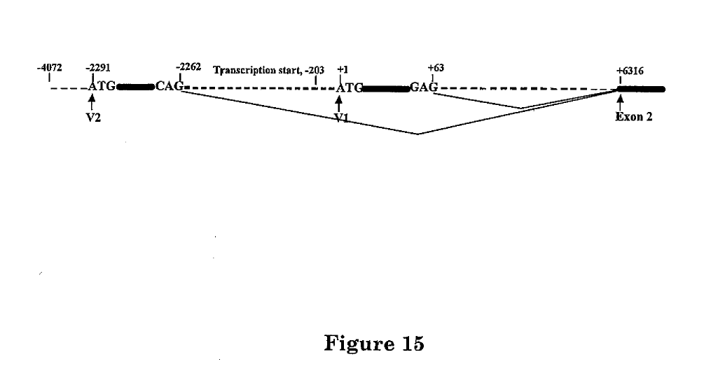

We realized existence of multiple MAT2β variants when we cloned its 4.1 kb 5′-flanking region (GenBank accession number AY223864). The two major variants differ only at their 5′ end. V1 encodes a 344 amino acid protein beginning MVGREKELSHFPGSCRLVE-. The alternatively spliced V2 utilizes a different first exon lying further upstream in the genomic sequence to encode a 323 amino acid isoform beginning MPEMPEDMEQ-. The reading frame for both variants converges after this point and is identical. FIG. 15 shows the genomic structure of MAT2βV1 and V2. Regulation of the V1 promoter (1.2 kb 5′-flanking) has been described (55). A critical Sp1 site at +9 was important for basal promoter activity in Cos-1 and Jurkat cells. Overexpression of V1 resulted in more rapid growth rate in liver cancer cell line HuH-7 (36). To our knowledge, other than the sequence submission to Genbank, V2 has not been described. We believe that these two variants differ in function and regulate proteins in addition to MAT II.

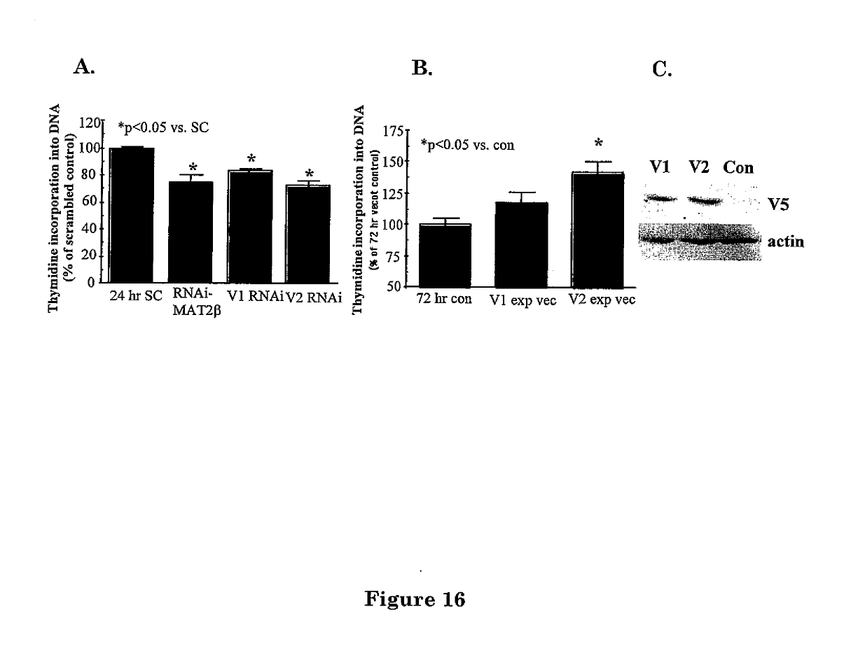

To better understand functional differences between the two MAT2β variants, we created vectors expressing either V1 or V2 using the mammalian expression vector pcDNA3.1D/V5-His/TOPO vector (Invitrogen). In this vector the MAT2β protein is fused to the viral V5 epitope and His tag at the carboxyl terminus. This allows detection of the expressed protein by Western blot analysis using an anti-V5 or anti-His antibody. We have transfected CaCo-2 cells to increase the expression of MAT2βV1 and V2 expression. We also created siRNA targeted against both MAT2β variants, V1 or V2 separately. After transient transfection of RKO cells with these siRNA constructs for 24 hrs, total MAT2β, V1 and V2 mRNA levels were reduced by 70%, 70%, and 75%, respectively. Specificity is assured, as RNAi against V1 had no influence on the mRNA level of V2 and vice versa. These cell lines were chosen because V2 expression is high in RKO cells and low in CaCo-2 cells (Table V). FIG. 16 shows the effect of varying MAT2β variant expression on cell growth in colon cancer cells. Cell growth was measured by 3H-thymidine incorporation into DNA. FIG. 16 shows that reducing MAT2β, V1 or V2 expression by 70-75% resulted in a 20-25% reduction in DNA synthesis (left panel). Overexpression of V2 (but not V1) in CaCo-2 cells increased DNA synthesis by 42% at 72 hrs (FIG. 16C). Since MAT2β is known to modulate the Ki of MAT II, we anticipated an increase in SAMe level with reduced MAT2β expression. However, there were minimal changes in SAMe levels (scrambled control=0.35±0.02, RNAi against both variants=0.40±0.02, RNAi against V1=0.33±0.03, RNAi against V2=0.36±0.02 nmol/mg protein, mean±SD from triplicates). On closer examination of the work by Martínez-Chantar et al, overexpression of MAT2β in HuH-7 cells lowered SAMe levels only when medium methionine concentration was raised from 15-100 μM to 1 mM (36). Hepatic methionine concentration is ˜40 μM (29) and colonic epithelial methionine concentration is likely similar. Our experiments used 100 μM L-methionine and suggest the effect of MAT2β on cell growth occurred independent of changes in SAMe level.

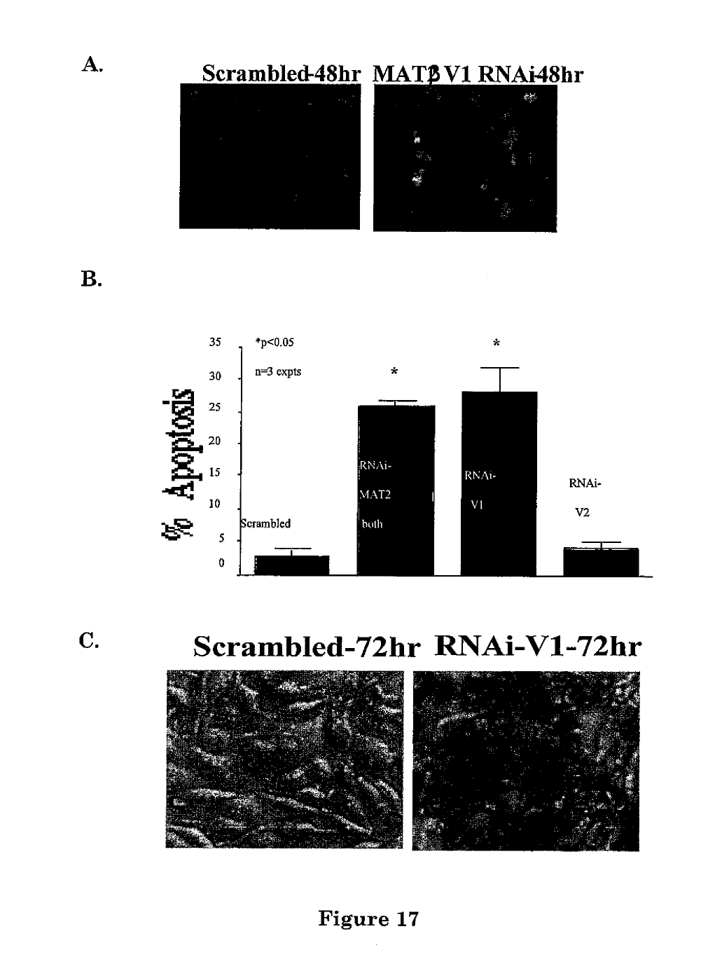

Not only does reduced MAT2β expression inhibit growth, it also promotes cell death. Cell death by apoptosis becomes obvious at 48 hrs after RNAi treatment targeted against both variants or V1, but not against V2 (FIG. 17). By 72 hrs, more than 75% of cells treated with V1 RNAi are dead. In RKO cells, the relative expression of MAT2βV1 to V2 is about 4:1. The observation that RNAi targeted against V2 does not cause cell death may be due to the fact that V1 is expressed at much higher level than V2 and can compensate for the loss of V2 (but not vice versa), or that V2 does not modulate apoptosis under these experimental conditions. However, V2 cannot act as a dominant-negative form of V1 since overexpression of V2 enhanced growth, not apoptosis.

### Effects of IGF-1, EGF and Leptin on MAT Expression and Proliferation in Colon Cancer Cell Lines

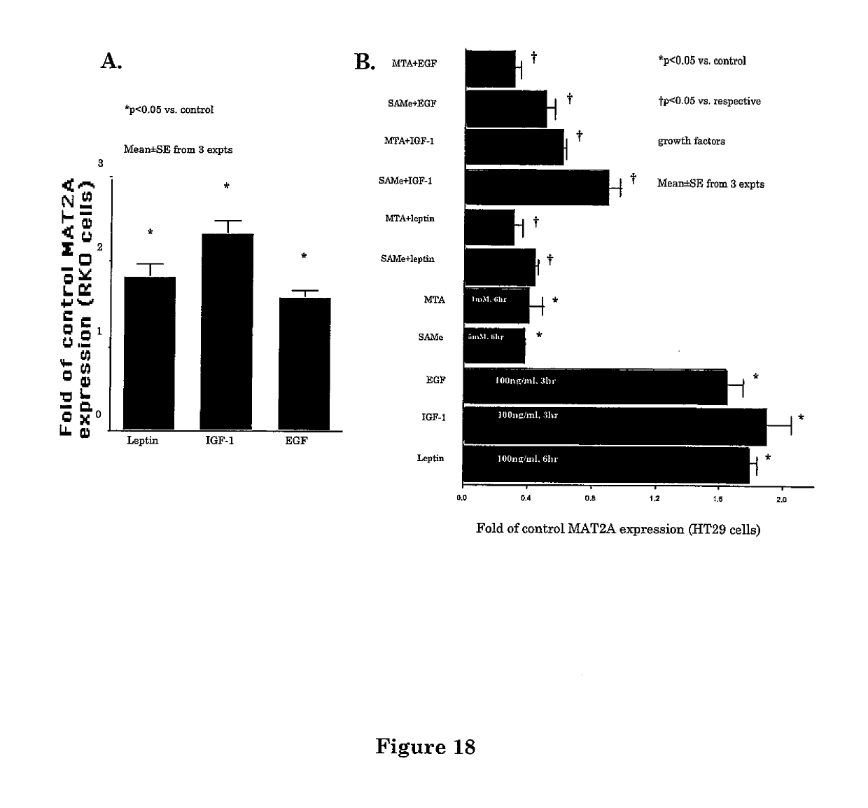

We examined the effect of three well-known growth factors that have been implicated colon cancer growth and invasion on MAT expression. Both higher levels of IGF-1 and IGF-1R have been shown in colon cancer (4, 7), and IGF-1R signaling plays an important role in tumor growth, angiogenesis and metastasis (4). Elevated IGF-1 levels also occur in insulin resistance and epidemiological studies support a link between insulin resistance and colon cancer (56). Similarly, increased EGF receptor signaling has also been demonstrated to provide growth advantage, correlate with colon cancer progression and metastatic potential (3, 8). Likewise, elevated leptin levels was found to be a risk factor for colon cancer in men (5), and leptin has been shown to be a mitogen in colon cancer cell lines (47), protect against sodium butyrate-induced apoptosis (57), and promote invasiveness of colon cancer cells (6). Leptin levels correlate with BMI, and obesity is a well-recognized risk factor for colon cancer (5). The leptin long receptor (OB-Rb) is expressed on the brush border of human enterocytes (58). To examine the effect of these growth factors on MAT expression and growth, colon cancer cells were plated at low confluence in serum free medium overnight, followed by treatment with leptin (100 ng/ml), IGF-1 (100 ng/ml), EGF (100 ng/ml), or vehicle for varying lengths of time. MAT expression was evaluated by real-time PCR and growth was evaluated by the MTT assay or BrDU incorporation after 24 hrs of treatment. We chose two cell lines, RKO and HT-29 for these studies because both are well characterized (p53 is intact in RKO but mutated in HT-29), and respond to growth and death modulatory stimuli (60, 47, 61). Leptin treatment of both RKO and HT29 cells induced a time-dependent increase in MAT2A expression, with maximum induction (doubling) occurring about 5 to 6 hours following treatment (FIG. 6). However, MAT2β (total and individual variants) expression was not affected by leptin treatment. IGF-1 and EGF treatment for 3 hours also induced MAT2A expression (but not MAT2β, data not shown) (FIG. 18). SAMe (5 mM) and MTA (1 mM) treatment for 6 hrs reduced the baseline expression of MAT2A in HT29 cells by 60% (FIG. 18B). In a separate time course study, SAMe and MTA lowered MAT2A expression by 10-20% and 60-65% after 1 hr and 3 hrs of treatment, respectively. Co-treatment of either SAMe or MTA with leptin (6 hrs) or EGF (3 hrs) prevented the ability of these growth factors to induce MAT2A. SAMe or MTA co-treatment (3 hrs) also blunted the IGF-1-mediated induction of MAT2A, with MTA exerting a much more potent effect than SAMe (FIG. 18B).

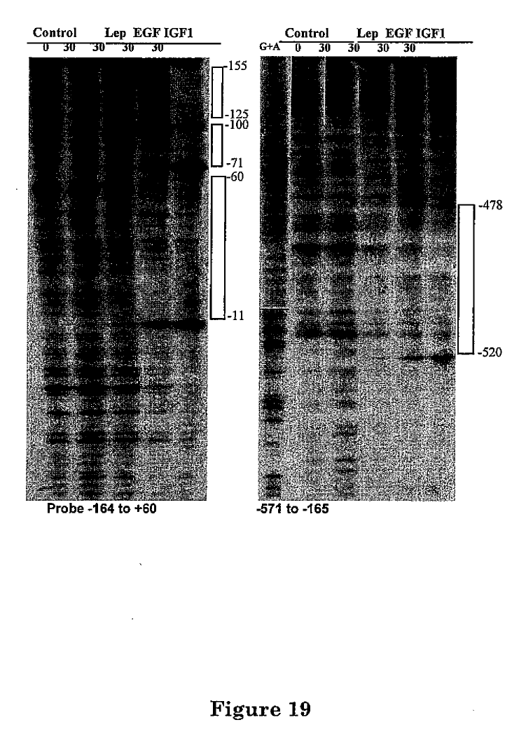

Next we examined the effect of these growth factors, SAMe and MTA on MAT2A promoter activity. HT29 cells were transfected with human MAT2A promoter constructs as we described (34). Treatment of HT29 cells with mitogens significantly increased, while treatment with SAMe or MTA inhibited MAT2A promoter activity (FIG. 7). Thus, the change in MAT2A mRNA level occurred due to a change in gene transcription. We have done DNase I footprinting in RKO cells treated with these mitogens for 6 hrs and FIG. 19 shows increased DNase I protected regions after treatment but there are differences in the regions protected among these mitogens, suggesting different molecular mechanisms may be involved.

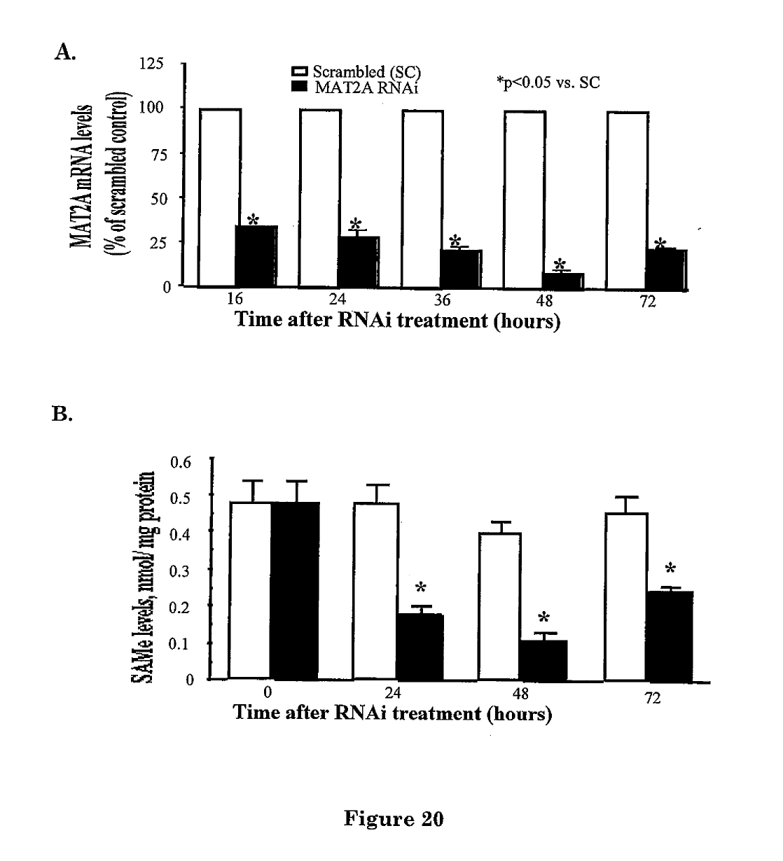

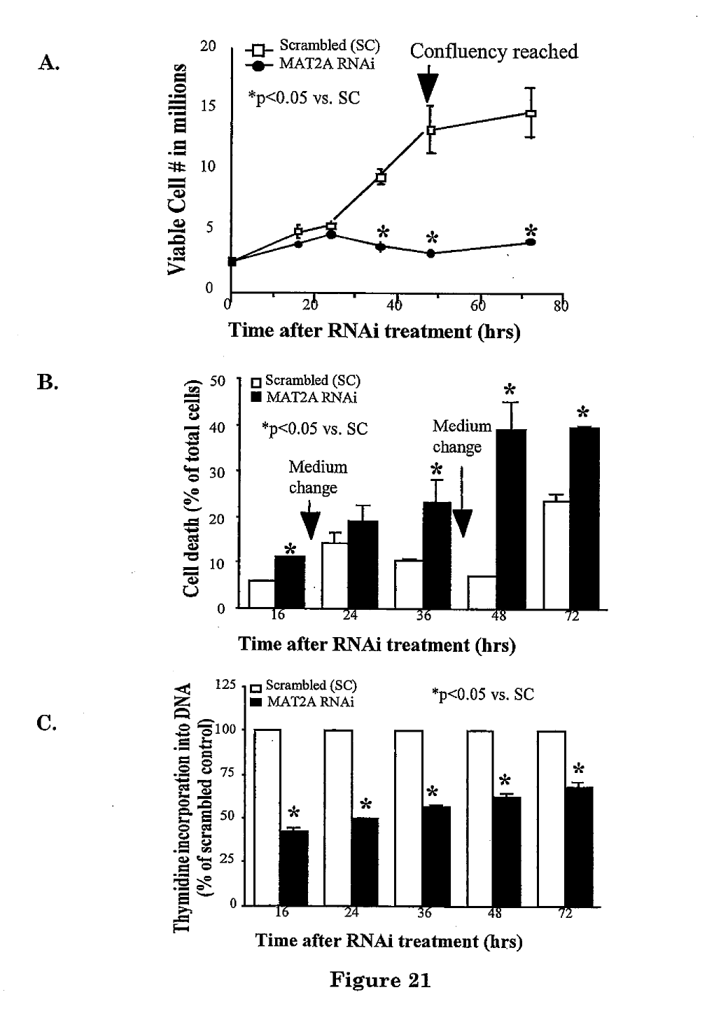

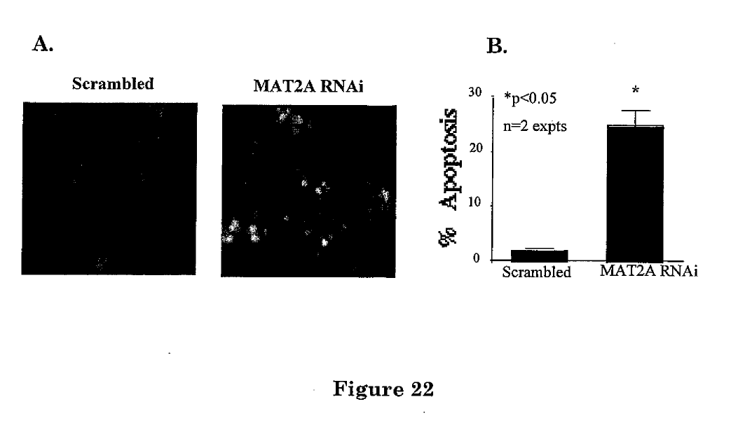

To see if an increase in. MAT2A was necessary for the mitogenic effect, we blocked the increase in MAT2A with siRNA. We chose a siRNA construct that resulted in 50% knockdown of MAT2A in 72 hrs. In the presence of this siRNA, mitogens were only able to raise MAT2A mRNA level to, baseline. The proliferative effect was significantly blunted and closely paralleled the effect on MAT2A expression (FIG. 11). Collectively these results suggest induction in MAT2A is required for the mitogenic effect. If MAT2A mRNA expression is reduced further, there is a dramatic effect on cell growth and death. The most potent siRNA reduced MAT2A mRNA level to <10% of baseline at 48 hrs (FIG. 20). SAMe levels fell to as low as 25% of SC (FIG. 20). FIG. 21 shows that there is complete cessation in cell growth following MAT2A RNAi treatment, increase in cell death (determined by trypan blue exclusion), and inhibition in DNA synthesis. To determine the form of cell death, cells were stained with Hoechst and Sytox green as described (62) and processed for Annexin V and propidium iodide staining followed by flow cytometry. The predominant type of cell death in the early time points (up to 24 hrs) is apoptosis (FIG. 22).

### Effect of MAT2A Knockdown on Polyamine Levels

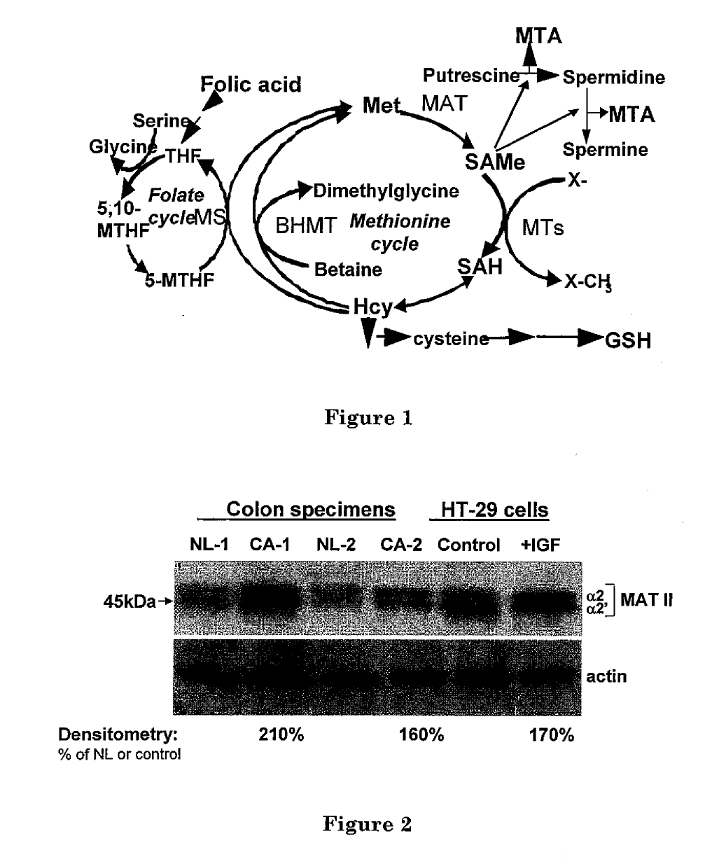

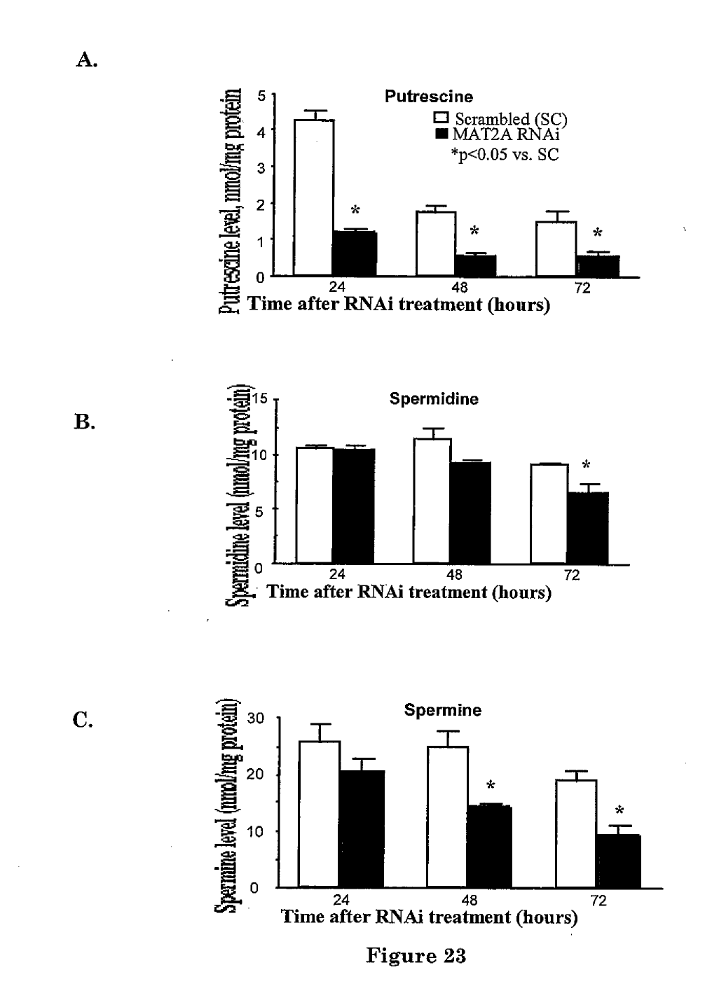

Polyamines are organic cations with multiple functions that are essential for the cell's survival (91). Mammalian cells have three polyamines: putrescine, spermidine and spermine. They interact with DNA (causing changes in chromatin structure), RNA and proteins and have antioxidant properties (63, 64). They regulate cell cycle progression and inhibition of polyamine synthesis leads to cell cycle arrest and apoptosis (65, 63). Cells have very complex regulatory mechanisms that control intracellular polyamine levels through biosynthesis, degradation and transport (67, 63). Putrescine is formed from the decarboxylation of ornithine, catalyzed by ornithine decarboxylase (ODC) and this combines with decarboxylated SAMe generates spermidine via spermidine synthase, and spermine through a second aminopropyltransferase reaction involving spermine synthase (FIG. 1). The catabolism of polyamines is essentially a reverse route of their biosynthesis and is catalyzed by spermidine and spermine N′-acetyltransferase and polyamine oxidase. Regulation occurs at multiple levels involving many of these enzymatic steps. Of interest, ODC expression is often induced and polyamine levels are higher in colon cancer and loss of APC function causes an increase in ODC activity and polyamine levels (64). In fact, clinical trials using difluoromethylornithine (DFMO), a specific inhibitor of ODC, are underway in the chemoprevention of colon polyps (64). Since MAT2A is responsible for biosynthesis of SAMe, a plausible hypothesis for cell cycle arrest and apoptosis with decreased MAT2A expression is reduced polyamine synthesis. In these recent experiments, we switched transfection protocol so that apoptosis following MAT2A knockdown did not occur until 48 hrs afterwards. FIG. 23 shows that all three polyamines exhibited a time-dependent decrease and putrescine was more than 70% depleted prior to onset of apoptosis. A similar fall in polyamine levels led to apoptosis in lymphocyte cell lines (63). Interestingly, ODC mRNA level fell by 40% at 24 hr and 50% at 48 hr. Importantly, MAT2β RNAi did not reduce ODC mRNA level. MAT2A RNAi treatment had little effect on the mRNA levels of spermidine synthase and spermine synthase and actually increased SAMe decarboxylase mRNA level at 48 hr. ODC is regulated at multiple levels and to our knowledge, regulation by SAMe content at the mRNA level has not been reported.

### Effect of SAMe and MTA on Growth and Death in Colon Cancer Cell Lines

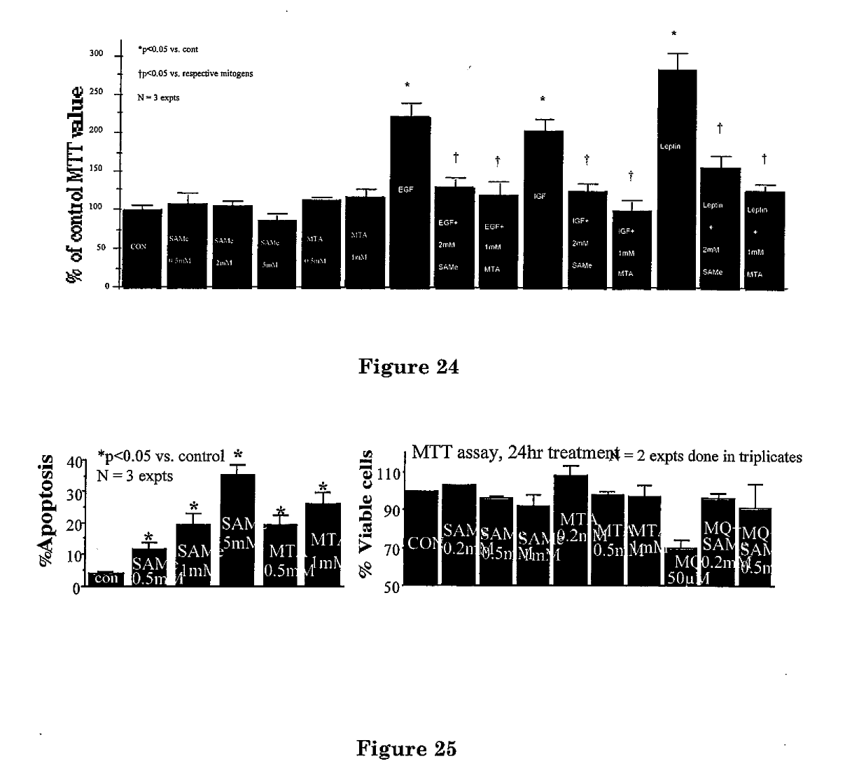

Similar to their growth inhibitory and pro-apoptotic effects in liver cancer cell lines (67), SAMe and MTA also induced the same changes in colon cancer cell lines. FIG. 24 shows that SAMe and MTA can block the mitogenic effect of growth factors. In these experiments the total treatment duration was limited to 16 hrs to avoid the apoptotic effect. By 24 hrs, SAMe and MTA induced apoptosis in a dose-dependent manner (FIG. 25, left panel). Apoptosis was measured by Hoechst staining or Annexin V and propidium iodide staining on flow cytometry. Importantly, SAMe (0.2 to 2 mM) and MTA (0.2 to 5 mM) did not cause any toxicity in NCM460 cells, the cell culture model of normal colonic epithelial cells (43). Furthermore, SAMe actually protected against menadione (MQ)-induced toxicity in NCM460 cells (FIG. 25, right panel).

NCM460 cell line was originally established in 1996 as the first non-malignant colonic epithelial cell line derived from the transverse colon (43). When first developed, cells formed both monolayer and in suspension, express villin and cytokeratin staining (43). NCM460 cells are non-tumorigenic, have similar features as normal human colonocytes in primary culture, exhibit vectoral Cl-transport and uptake mechanisms for various vitamins (43, 68-70). The NCM460 cells in monolayer have features of transverse colon crypt cells (69). We treated these cells with mitogens as above and found very little change in MAT2A expression under the same experimental conditions. SAMe and MTA inhibited MAT2A expression but to a lesser degree (5 mM SAMe or 1 mM MTA for 6 hrs reduced MAT2A expression by 40% in two separate experiments, as compared to 60% reduction in colon cancer cell lines).

The fact that both reduced SAMe level (by MAT2A RNAi) and exogenous SAMe treatment can lead to apoptosis may seem like a paradox. However, a bell shape response could, in fact, be a general paradigm in biological systems. For example, both polyamine depletion and excess can cause apoptosis. An alternative explanation is that at pharmacological doses of SAMe, the effect is actually mediated by its metabolite MTA. SAMe is unstable, converts to MTA spontaneously and in the polyamine pathway. While SAMe is a methyl donor and precursor of polyamines, MTA inhibits methylation and polyamine synthesis (53). Indeed, the half-life of SAMe (1 to 5 mM) is 12 hrs while the rate of conversion from SAMe to MTA is 0.013 mM per hour per mM SAMe in culture medium at 37° C. In contrast, MTA is highly stable (no change in 24 hrs under the same condition). Table III shows intracellular SAMe and MTA levels after treatment of HT-29 cells with mitogens, SAMe or MTA. All Mitogens increased intracellular SAMe levels by 16 hrs, as did SAMe and MTA. MTA can raise SAMe level because it is converted back to SAMe via the methionine salvage pathway (71). Intracellular MTA levels increased dramatically after SAMe treatment (19-fold with 5 mM SAMe for 16 hrs). Thus, while mitogens increased SAMe within physiological levels to support polyamine synthesis and growth, pharmacological doses of SAMe will inhibit growth most likely because it increased MTA. After treatment with mitogens, SAMe or MTA for 6 hrs, putrescine levels increased in the mitogen treated groups (Table IV). However, SAMe and MTA treated groups had lower putrescine levels and MTA treated group also had lower spermidine and spermine levels (Table IV). MTA treatment for 6 hrs lowered the mRNA levels of ornithine decarboxylase (ODC), spermidine and spermine synthase by 30%. This was not expected since MTA was thought to inhibit enzyme activity only (71, 53).

### Effects of Oral SAMe and MTA Supplementation on Intestinal SAMe Levels and MAT2A Expression

The effect of SAMe (75 or 150 mg/kg/d) and MTA (75 mg/kg/d) supplementation in water for six days on the intestinal MAT gene expression and SAMe/MTA levels in wild type mice was tested. The dose of SAMe was chosen based on the fact that 200 mg/kg/day is the dose shown to prevent HCC formation in rats (18) and adult human therapeutic dose is 1.2 to 3.6 gm/day taken orally (44). SAMe and MTA were well tolerated and animals drank comparable amounts of water and gained similar amounts of weight as controls. The changes in proximal small intestine and colon were examined because these regions should receive the highest and lowest amounts of SAMe and MTA, respectively. Table VI shows that intestinal levels of SAMe increased in a dose-dependent fashion by SAMe supplementation, especially in proximal small intestine (800% of control). Even colonic mucosa had nearly 200% increase in SAMe level. Intestinal MTA levels were below detection limit. Importantly, both high dose SAMe and MTA decreased MAT2A expression in proximal small intestine by 20% after only six days.

TABLE VI
Intestinal SAMe levels and MAT2A expression with
SAMe or MTA treatment

| | Proximal small intestine | | Colon | |
|---|---|---|---|---|
| | SAMe | MAT2A | SAMe | MAT2A |
| ID | level | expression | level | expression |
| Control | 0.28 ± 0.01 | 100 ± 0 | 0.47 ± 0.02 | 100 ± 0 |
| SAMe | 0.58 ± 0.16 | 99 ± 2 | 0.97 ± 0.10** | 128 ± 20 |
| (75 mg/kg/d) | | | | |
| SAMe | 2.29 ± 0.55* | 81 ± 4† | 1.21 ± 0.14** | 85 ± 10 |
| (150 mg/kg/d) | | | | |
| MTA | 0.63 ± 0.09* | 82 ± 2† | 0.72 ± 0.04** | 87 ± 20 |
| (75 mg/kg/d) | | | | |

SAMe levels are in nmol/mg protein, MAT2A expression is expressed as % of control MAT2A mRNA levels. Wild type C57/B6 mice were given SAMe or MTA in drinking water daily for six days. Results are mean ± SE from four mice per group.
*p < 0.05,
**p < 001,
†p < 0.001 vs. control group.

### SAMe and MTA Induces Apoptosis in Colon Cancer Cells

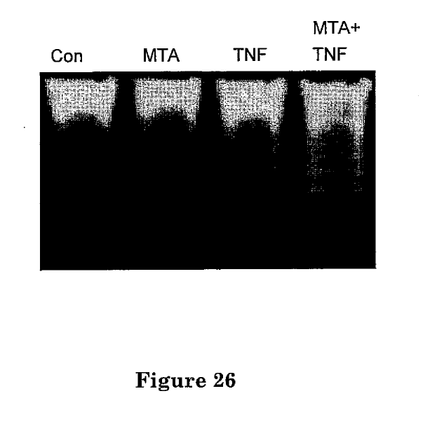

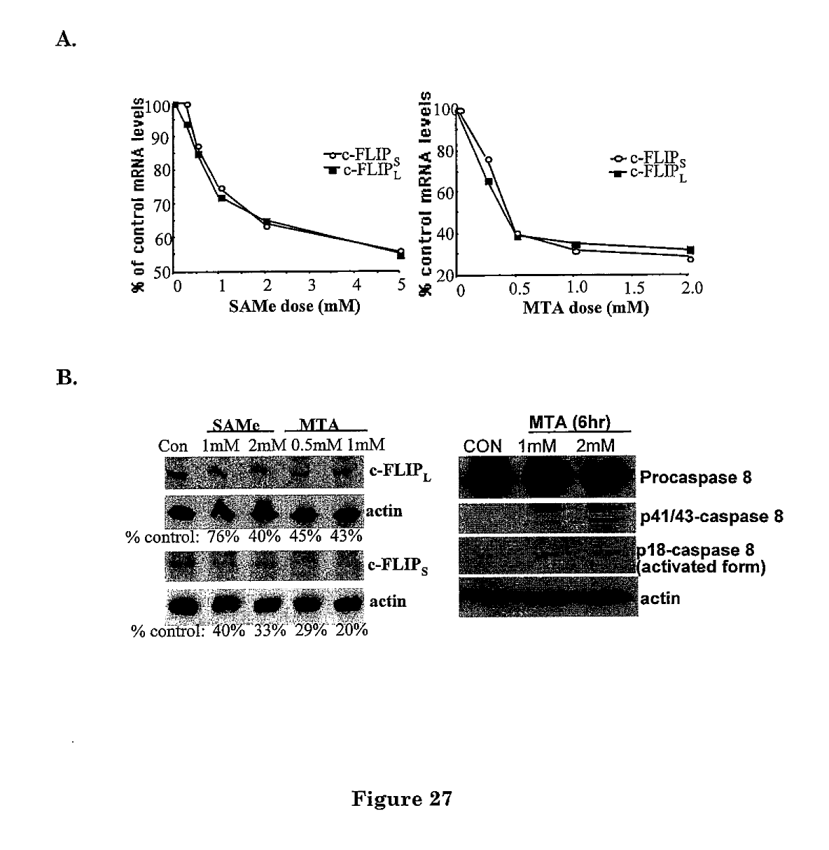

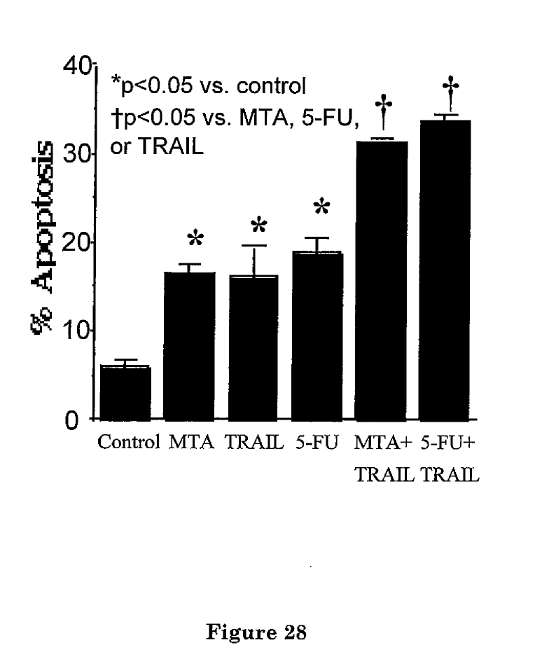

To study the mechanism of exogenous SAMe and MTA-induced apoptosis, Oligo GEArray Human Apoptosis Array (CAT# OHS-012, Superarray) was used. The effect of SAMe (2 mM) and MTA (1 mM) treatment of HT-29 cells for 6 hrs on gene expression was examined. Several genes were altered but c-FLIP, which was down-regulated particularly by MTA was of interest, since this is a protein that is structurally related to procaspase-8 and -10 but lacks enzymatic activity (72). Two c-FLIP proteins exist, a 26-kDa short form (c-FLIP_{S}) and a 55-kDa long form (c-FLIP_{L}) (72). The short form is a proteolytic product of the long form and both isoforms are anti-apoptotic. Furthermore, increased expression of the long isoform can resist TNFβ-related apoptosis-inducing ligand (TRAIL)-mediated apoptosis (73-77). Interestingly, c-FLIP is overexpressed in colon cancer (77) and the long form has been shown to inhibit chemotherapy-induced colorectal cancer cell death (78). The results of the microarray using real-time PCR have been verified and show that SAMe and MTA lowered c-FLIP mRNA level to 70±2% and 47±4% of control, respectively (results are mean±SE from 3 experiments, p<0.005 in both). Consistent with this, MTA potentiated TNFα-induced apoptosis in HT-29 cells (FIG. 26). SAMe and MTA also lowered c-FLIP expression in RKO cells, with concomitant caspase 8 activation (FIG. 27) and sensitized RKO cells to TRAIL-induced apoptosis (FIG. 28). There is a growing interest in using TRAIL in chemotherapy as TRAIL appears to exert selective pro-apoptotic effect in transformed cells but not normal cells (76). 5-fluorouracil (5-FU) was shown to sensitize colon cancer cells to TRAIL-induced apoptosis by lowering c-FLIP expression (76). Our results show MTA can do the same and likely without the side effects of 5-FU.

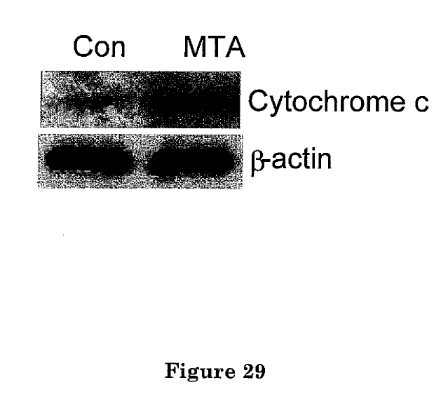

SAMe and MTA induce apoptosis in liver cancer cells by the mitochondrial pathway (14). FIG. 29 shows that in colon cancer cells, mitochondria are also involved as cytochrome c release is evident. In liver cancer cells, SAMe and MTA induced selectively expression of Bcl-xs, a pro-apoptotic protein (67). We examined whether the same occurs in HT-29 cells. After 6 hrs of MTA (1 mM) treatment, Bcl-x_{L} mRNA level fell by 53% (47±4% of control, results are mean±SE from 4 experiments, p<0.0005). At this time point, SAMe (2 mM) did not have an effect and there is also no effect on Bcl-x_{S} mRNA level. These results differ from the effect of these agents in liver cancer cells where Bcl-x_{L} was not affected. Collectively, these data suggest these agents when used at pharmacological doses, induce apoptosis by affecting the expression of Bcl-2 family members influencing the apoptotic pathway involving the mitochondria and sensitize the colon cancer cells to death receptor mediated apoptosis by lowering c-FLIP expression. Importantly, SAMe (up to 2 mM) and MTA (up to 5 mM) do not cause apoptosis in NCM460 cells, the only available normal colonic epithelial cell line. This makes these agents particularly attractive in targeting colon cancer cells.

### MAT and SAMe in Adenomatous Polyps and in Response to SAMe and MTA Supplementation

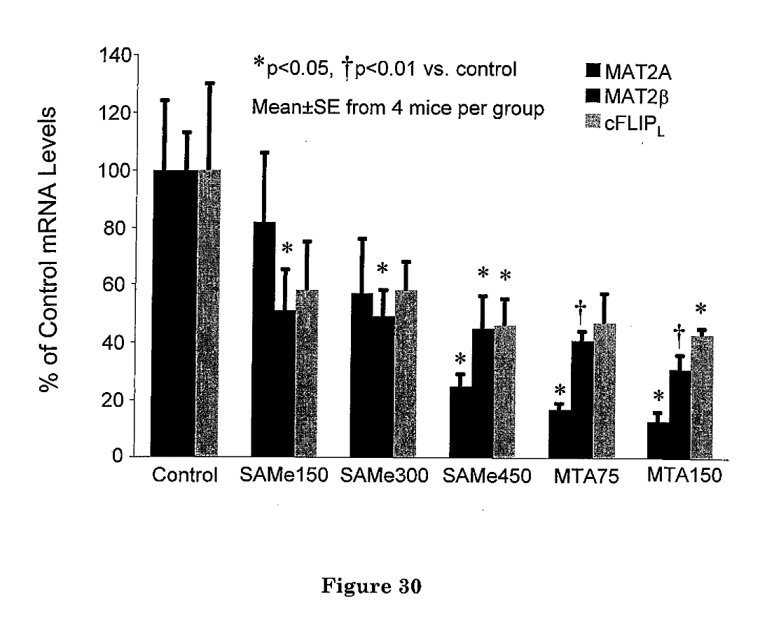

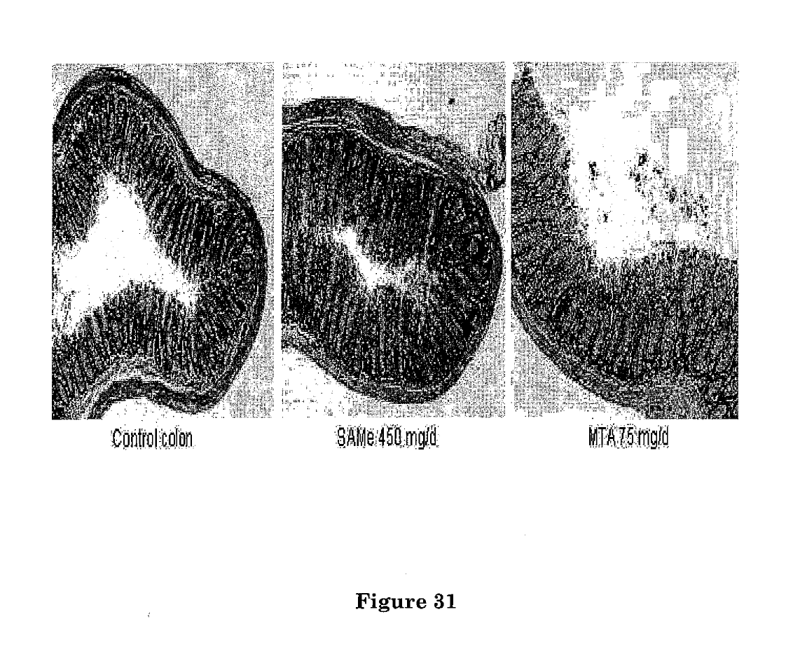

To see if the in vitro observation was applicable in vivo, nine paired samples of adenomatous polyps and adjacent uninvolved mucosa from three Min mice were obtained. Min mice are heterozygous for the multiple intestinal neoplasia (Min) mutation of the adenomatous polyposis coli (APC) gene (79). The Min mouse is generally agreed to be a useful model for the study of early stages of intestinal cancer as the APC gene is usually inactivated early in the carcinogenic process in man, both in sporadic colorectal cancer and in familial polyposis coli (92). MAT2A mRNA levels were higher in all polyps (338±93%, range 132% to 909%, expressed as percentage of matched normal tissue mRNA level, p<0.05). SAMe levels measured in four polyps (average MAT2A mRNA levels were 200±50% of normal) were also higher than adjacent normal tissues (180±8% of normal, p<0.05). This makes SAMe and MTA attractive as chemopreventive agents if they can suppress MAT2A gene expression in vivo. To test this possibility, the effect of SAMe (75 or 150 mg/kg/d) and MTA (75 mg/kg/d) supplementation in water for 6 days on the intestinal MAT gene expression and SAMe/MTA levels in wild type mice was examined. The dose of SAMe was chosen based on the fact that 200 mg/kg/day is the dose shown to prevent HCC formation in rats (18) and adult human therapeutic dose is 1.2 to 3.6 gm/day taken orally (44). SAMe and MTA were well tolerated and animals gained similar amount of weight as controls. The intestine were divided into four sections—proximal, mid and distal small intestine and colon. Mucosa was scraped off and processed for SAMe/MTA measurement as well as RNA extraction. Intestinal levels of SAMe increased in a dose-dependent fashion by SAMe supplementation, especially in proximal small intestine (up by 8-fold) (Table VI). Even colonic mucosa had nearly 3-fold increase in SAMe level. Importantly, both SAMe (150 mg/kg/d) and MTA (75 mg/kg/d) decreased MAT2A expression in proximal small intestine by 20% after only 6 days (93). We have further expanded the dose finding experiment to as much as 450 mg/kg/d SAMe and 150 mg/kg/d MTA. FIG. 30 shows a dose-dependent inhibition in MAT2A mRNA level by SAMe with 75% inhibition achieved at the highest dose. MTA was already optimal at 75 mg/kg/d (85% inhibition). Interestingly, MAT2β expression was also inhibited by 50% at 150 mg/kg/d SAMe and 60% at 75 mg/kg/d MTA. Importantly, both SAMe and MTA inhibited cFLIP_{L} expression, confirming data obtained in vitro (FIG. 27). SAMe and MTA caused no apparent toxicity as weight gain and water consumption were similar to controls. Lack of toxicity was also demonstrated histologically (FIG. 31).

### MTA Treatment for Chemoprevention of Aberrant Crypt Foci Formation in Min Mice

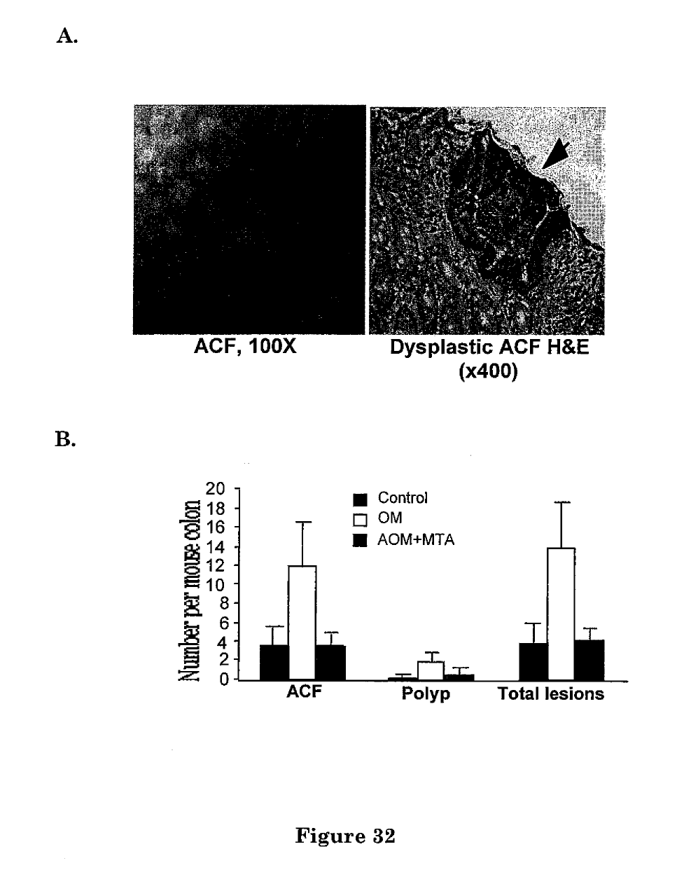

Encouraged by our in vitro and in vivo data, we examined whether MTA supplementation in drinking water can prevent the formation of aberrant crypt foci (ACF) in Min mice. Azoxymethane (AOM), an intermediate in the metabolism of 1,2-dimethylhydrazine (DMH), is a colorectal-specific procarcinogen like DMH (80). ACF are putative preneoplastic lesions that are induced in the colon of carcinogen-treated rodents and are present in humans with a high risk of colon cancer development (81). ACF are characterized by elevated crypts, thicker epithelial cell lining, and stain more intensely with methylene blue (80). Classically ACF are elevated above the mucosa (81). Interestingly, Min mice have dysplastic ACF that are not raised (termed ACFₘᵢₙ) and are picked up by methylene blue staining and transillumination (81). However, when Min mice are treated with AOM, both classical ACF and ACF_{Min} are greatly induced and it appears that the ACF_{Min} are the precursor of adenoma (82). The effect of MTA in AOM treated Min mice were examined because dysplastic ACF and tumors were induced by 6 to 10 weeks after first AOM treatment (82). This relatively short duration makes this model particularly attractive. In our preliminary experiment, 9 Min mice were divided into 3 groups (n=3 each), control, AOM, and AOM+MTA. AOM was given at 5 mg/kg s.c. once/week×2 starting at 3 to 4 weeks of age and MTA (75 mg/kg/d) was given in drinking water. Mice were sacrificed 6 weeks after the first AOM treatment. We followed the procedure described for fixation and scoring of ACF and tumors polyps, >12 crypts) (82). FIG. 32 shows typical appearance of ACF_{Min} under the microscope. AOM treatment tended to increase the number of ACFs as compared to the control group (p=0.1) and MTA treatment tended to prevent the increase (p=0.1). The difference is not statistically significant due to low number of animals. Majority of the ACFs found were of the ACF_{Min} type, showing dysplasia on H&E.

The results showed increased MAT2A and MAT2β (mostly variant 2) mRNA levels in resected colon cancer specimens as compared to adjacent normal tissue. Epidermal growth factor (EGF), insulin-like growth factor-1 (IGF-1) and leptin, mitogens implicated in the growth and invasiveness of colon cancer, increased MAT2A mRNA levels, MAT2A promoter activity, and growth of colon cancer cell lines RKO and HT29. Lowering MAT2A expression by RNAi reduced growth and blocked the mitogenic effects of these growth factors. SAMe and MTA lowered the expression of MAT2A in RKO and HT29 cells, and prevented the ability of growth factors to induce MAT2A expression and cell proliferation. Furthermore, SAMe and MTA inhibited growth of colon cancer cells by inducing apoptosis.

### Discussion

MAT is a critical cellular enzyme because it catalyzes the only reaction that generates SAMe, the biological methyl donor and precursor for polyamines. In polyamine synthesis SAMe is decarboxylated to form S-adenosylmethionineamine. The propylamine moiety of S-adenosylmethionineamine is then donated to putrescine, to form spermidine and a first molecule of MTA. Spermidine is then converted into spermine by the addition of one more propylamine group from S-adenosylmethionineamine which generates a second molecule of MTA (9). In mammalian liver, MAT1A is a marker for the differentiated or mature liver phenotype, while MAT2A is a marker for rapid growth and de-differentiation (9). Recently, MAT2β has also been shown to be induced in human liver cancer and to provide a growth advantage (36). While much is known about the role of dysregulation of MAT genes in liver disease and cancer, very little is known about regulation of these MAT genes outside of the liver, and virtually nothing is known about the regulation or dysregulation of these genes in colon cancer. Although MAT is responsible for generation of SAMe, which may enhance growth through the polyamine pathway, SAMe treatment of normal and malignant hepatocytes actually inhibits growth (13, 22). Whether SAMe or its metabolite MTA can influence growth in colon cancer cells has not been examined.

Although no study has examined the role of SAMe in colon cancer pathogenesis, by comparison, there is an abundant literature on the role of folate on colon cancer risk. It is well recognized that folic acid deficiency correlates with increased risk of colorectal cancer (83). One of the consequences of folic acid deficiency is SAMe deficiency as folic acid is involved in the remethylation of homocysteine to methionine, the precursor of SAMe (9). However, folic acid supplementation can either prevent or exacerbate intestinal tumorigenesis, depending on the timing of supplementation (83). This may be related to the fact that folic acid is also a precursor for nucleotides. This also makes the supplementation of folic acid in colon cancer complicated. Interestingly, low methionine intake in the diet, which would be expected to lower SAMe levels, is also a recognized risk factor for colon cancer (84). SAMe is now widely available in the United States as a nutritional supplement and is largely free of side effects (9). Aspects of the present invention were to examine whether the expression of MAT genes are altered in colon cancer, whether mitogens implicated in colon cancer pathogenesis can influence MAT genes expression, and whether SAMe can modulate this response.

MAT2A mRNA levels are higher in 12 of 13 colon cancer specimens. This resulted in higher MAT II protein levels. In contrast to liver cancer where MATH is also induced, it is mostly unchanged in colon cancer. To see if this pathway is important in the early state of intestinal neoplasia, adenomatous polyps from Min mice, which are heterozygous for the multiple intestinal neoplasia (Min) mutation of the adenomatous polyposis coli (APC) gene were examined (46, 85). The Min mouse is generally agreed to be a useful model for the study of early stages of intestinal cancer as the APC gene is usually inactivated early in the carcinogenic process in man, both in sporadic colorectal cancer and in familial polyposis (85). Indeed MAT2A mRNA levels are higher in all polyps.

The effect of three well-known growth factors on the expression of MAT genes in two different colon cancer cell lines were examined. In order to make sure the response is not unique to a particular cell line. Each of these growth factors has been implicated in the pathogenesis of colon cancer. All have been demonstrated to be mitogenic in colon cancer cell lines (4, 8, 6). Higher levels of the growth factor and/or its receptor have also been found in colon cancer (4, 7, 3, 8, 5, 6). All three growth factors induced MAT2A gene expression but had little influence on MATH in both colon cancer cell lines. SAMe and its metabolite MTA, lowered MAT2A expression and largely prevented the induction of MAT2A by these growth factors. Investigation was extended to MTA because MTA is a product of SAMe metabolism in the polyamine pathway as well as non-enzymatic hydrolysis (53). Previous results have shown that SAMe's effect on cell growth and gene expression can be mimicked by MTA (14, 15, 87, 88). In contrast to SAMe, MTA inhibits methylation and polyamine synthesis (53, 86). Thus, additional insights can be gained by comparing the effect of these two agents. While not wanting to be bound by theory, the fact that MTA is more potent than SAMe for many of SAMe's effects suggests that many of SAMe's action may in fact be mediated via MTA.

Transcriptional regulation of human MAT2A in human liver cancer cells have been characterized and found four functional cis-acting elements and their corresponding transcription factors to contribute to the up-regulation of MAT2A in human liver cancer (34, 32). In colon cancer cells, all three growth factors induced the MAT2A promoter activity to levels comparable to their effects on endogenous MAT2A expression. Both SAMe and MTA lowered the MAT2A promoter activity, also to levels comparable to their effects on the endogenous gene expression. These results support the notion that the effect lies at the transcriptional level. Specificity is assured as SAMe and MTA had no effect on the human GSS promoter activity.

To address whether the increase in MAT2A is causally linked to increased growth, the following two approaches were taken. One was to block mitogen-mediated MAT2A induction by SAMe and MTA. Both agents prevented the mitogenic effect of these growth factors. However, given that these agents may have other effects besides modulating MAT2A expression, the inventor also took a more direct approach, namely modulating the level of MAT2A expression using RNAi. A marked lowering of MAT2A expression (using RNAi#1, which lowered MAT2A expression by 90%) actually resulted in increased apoptosis by 24 hrs. This result is consistent with the fact that MAT is an essential gene required for cell's survival and agrees with the inventor's previous results in liver cancer cell line HuH-7 where anti-sense directed against MAT2A led to cell death (13). To avoid the toxic effect of potent RNAi, a construct that lowered MAT2A expression by only 50% was chosen. Using this strategy, the level of MAT2A expression after treatment with leptin, IGF-1 or EGF was similar to untreated controls. Importantly, the effect on growth closely paralleled that of MAT2A expression, strongly supporting the notion that increased MAT2A expression is required for the growth factors to induce their mitogenic effect.

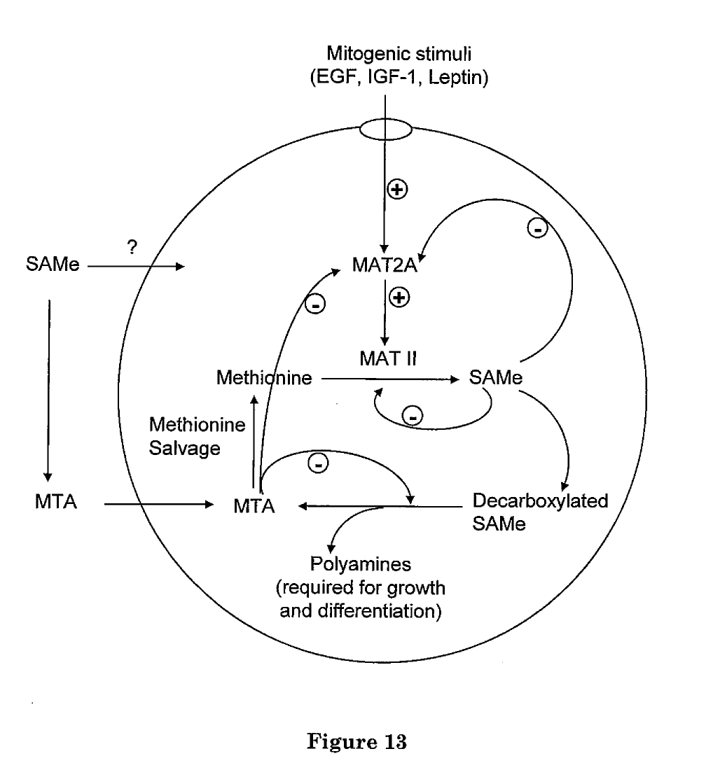

An increase in MAT2A without a change in MATH should result in higher MAT II activity and SAMe level (9). SAMe levels are higher in colon cancer specimens and polyps from Min mice. This is likely to provide the needed precursor for polyamines in growing cells. Consistently, all mitogens raised intracellular SAMe and polyamine levels and this is likely to be the mechanism for increased growth. The observation that increased SAMe level via induction of MAT2A induces growth while exogenous SAMe treatment inhibits growth may seem like a paradox. However, a bell shape response could, in fact, be a general paradigm in biological systems—too little and too much are both bad. An alternative explanation is that at pharmacological doses of SAMe, the effect is actually mediated by its metabolite MTA. SAMe is unstable, converts to MTA spontaneously and in the polyamine pathway. While SAMe is a methyl donor and precursor of polyamines, MTA inhibits methylation and polyamine synthesis (53). Indeed, high dose SAMe treatment raised intracellular MTA levels by much higher magnitudes than SAMe levels itself. Thus, while mitogens increased SAMe within physiological levels to support polyamine synthesis and growth, pharmacological doses of SAMe will inhibit growth most likely because it increased MTA. These findings are summarized in FIG. 13.

To see if the in vitro findings using pharmacological doses of SAMe are relevant in vivo, mice were treated with oral supplementation of pharmacological doses of SAMe or MTA. Importantly, these agents were well tolerated under the experimental protocol and raised intestinal SAMe levels while lowering MAT2A mRNA levels. Given that these are normal mice, the effect may be even greater in the setting of Min mice.

The influence of cellular SAMe level on growth is very different in colon cancer cells as compared to normal hepatocytes. In hepatocytes, due to presence of MAT1A, an increase in MAT2A actually results in lowering of SAMe level (13). This is because of the difference in the isoenzyme kinetics and the ability of SAMe to feedback inhibit MAT II but not MAT I or III (9). It has been shown that a fall in cellular SAMe level occurs prior to increased liver growth (12, 18). This then releases the inhibitory effect SAMe has on growth factors such as hepatocyte growth factor and allows the liver to regenerate (22). The situation in colon cancer cells is more similar to T-lymphocytes, where SAMe level rises during activation (37). While an increase in SAMe level facilitates growth of colon cancer cells, a fall in SAMe level, which can occur with folate deficiency or low methionine intake, can result in global DNA hypomethylation (83). This can lead to increased expression of oncogenes and chromosomal instability (89). In liver, chronic SAMe deficiency results in spontaneous development of hepatocellular carcinoma (40). Thus, the influence of SAMe should be considered in different settings—deficiency, physiological increase, and pharmacological use.

These results demonstrate that MAT2A expression is increased in human colon cancer and in response to growth factors that have been implicated in colon cancer pathogenesis. The mitogenic response of the growth factors closely parallel their ability to induce MAT2A expression. SAMe and MTA down-regulate MAT2A expression when used at pharmacological doses and block the ability of the growth factors to induce MAT2A and exert the mitogenic response. Collectively, these results support an important role for MAT2A in colon cancer pathogenesis and indicate that SAMe and MTA can be effective as chemopreventive agents in colon neoplasia.

Obviously, many modifications and variation of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

The following references are cited herein. The entire disclosure of each reference is relied upon and incorporated by reference herein.
1. Greenlee, R. T., T. Murray, S. Bolden et al. Cancer Statistics. CA Cancer J. Clin. 50:7-33, 2000.
2. Piard, F., C. Chapusot, A. Ecarnot-Laubriet, T. Ponnelle, and L. Martin. Molecular markers of heterogeneity in colorectal cancers and adenomas. Eur J. Cancer Prevention 11:85-97, 2002.
3. Roberts, R. B., L. Min, M. K. Washington, S. J. Olsen, S. H. Settle, R. J. Coffey, and D. W. Threadgill. Importance of epidermal growth factor receptor signaling in establishment of adenomas and maintenance of carcinomas during intestinal tumorigenesis. PNAS 99:1521-1526, 2002.
4. Reinmuth, N., F. Fan, W. Liu, A. A. Parikh, O. Stoeltzing, Y. D. Jung, C. D. Bucana, R. Radinsky, G. E. Gallick, and L. M. Ellis. Impact of insulin-like growth factor receptor-1 function on angiogenesis, growth, and metastasis of colon cancer. Lab. Invest. 82:1377-1389, 2002.
5. Stattin P, Lukanova A, Biessy C, Soderberg S, Palmqvist R, Kaaks R, Olsson T, Jellum E. Obesity and colon cancer: does leptin provide a link? Int. J. Cancer 2004; 109:149-152.
6. Attoub S, Noe V, Pirola L, Bruyneel E, Chastre E, Mareel M, Wymann M P, Gespach C. Leptin promotes invasiveness of kidney and colonic epithelial cells via phosphoinositide 3-kinase, Rho- and Rae-dependent signaling pathways. FASEB J 2000; 14:2329-2338.
7. LeRoith D, Roberts Jr. CT. The insulin-like growth factor system and cancer. Cancer Letts. 2003; 195:127-137.
8. Yokoi J, Thaker P H, Yazici S, Rebhun R R, Nam D H, He J, Kim S J, Abbruzzese J L, Hamilton S R, Fidler I J. Dual inhibition of epidermal growth factor receptor and vascular endothelial growth factor receptor phosphorylation by AEE788 reduced growth and metastasis of human colon carcinoma in an orthotopic nude mouse model. Cancer Res 2005; 65:3716-3725.
9. Lu S C, Mato J M. Role of Methionine Adenosyltransferase and SAMe in Alcohol-Associate Liver Cancer. Alcohol 2005; 35:227-234.
10. Kotb M, Mudd S H, Mato J M, Geller A M, Kredich N M, Chou J Y, Cantoni G L. Consensus nomenclature for the mammalian methionine adenosyltransferase genes and gene products. Trends in Genetics 1997; 13:51-52.
11. Cai J, Sun W M, Hwang J, Stain S, Lu S C. Changes in S-adenosylmethionine synthetase in human liver cancer: molecular characterization and significance. Hepatology 1996; 24:1090-1097.
12. Huang Z, Mao Z, Cai J, Lu S C. Changes in methionine adenosyltransferase during liver regeneration in the rat. Am. J. Physiol. 1998; 38:G14-21.
13. Cai J, Mao Z, Hwang J, Lu S C. Differential expression of methionine adenosyltransferase genes influences the rate of growth of human hepatocellular carcinoma cells. Cancer Res. 1998; 58:1444-1450.
14. Ansorena E, García-Trevijano E R, Martinez-Chantar M L, Huang Z Z, Chen L X, Mato J M, Iraburu M, Lu S C, Avila M A. S-adenosylmethionine and methylthioadenosine are anti-apoptotic in cultured rat hepatocytes but pro-apoptotic in human hepatoma cells. Hepatology 2002; 35:274-280.
15. Yang H P, Sadda M R, Li M, Zeng Y, Chen L X, Bae W J, Ou X P, Runnegar M T, Mato J M, Lu S C. S-Adenosylmethionine and its metabolite induce apoptosis in HepG2 cells: role of protein phosphatase 1 and Bcl-x_{S}. Hepatology 2004; 40:221-231.
16, Garcea, R., L. Daino, R. M. Pascale, M. M. Simile, M. Puddu, S. Frasseto, P. Cozzolino, et al. Inhibition of promotion and persistent nodule growth by S-adenosyl-L-methionine in rat liver carcinogenesis: role of remodeling and apoptosis. Cancer Res 49:1850-1856, 1989.
17. Pascale, R. M., M. M. Simile, M. R. De Miglio, A. Nufris, L. Daino, M. A. Seddaiu, P. M. Rao, et al. Chemoprevention by S-adenosyl-L-methionine of rat liver carcinogenesis initiated by 1,2-dimethylhydrazine and promoted by orotic acid. Carcinogenesis 16:427-430, 1995.
18. Pascale, R. M., M. M. Simile, G. Satta, M. A. Seddaiu, L. Daino, G. Pinna, M. A. Vinci, L. Gaspa, and F. Feo. Comparative effects of L-methionine, S-adenosyl-L-methionine and 5′-methylthioadenosine on the growth of preneoplastic lesions and DNA methylation in rat liver during the early stages of hepatocarcinogenesis. Anticancer Res. 11:1617-1624, 1991.
19. Pascale, R. M., V. Marras, M. M. Simile, L. Daino, G. Pinna, S. Bennati, M. Carta, M. A. Seddaiu, G. Massarelli, and F. Feo. Chemoprevention of rat liver carcinogenesis by S-adenosyl-L-methionine: A long-term study. Cancer Res. 52:4979-4986, 1992.
20. Martínez-Chantar, M. L., F. J. Corrales, A. Martínez-Cruz, E. R. García-Trevijano, Z. Z. Huang, L. X. Chen, G. Kanel, M. A. Avila, J. M. Mato, SC. Lu. Spontaneous oxidative stress and liver tumors in mice lacking methionine adenosyltransferase 1A. FASEB J 10:1096, Jun. 7, 2002.
21. Garcia-Trevijano, E. R., M. L. Martínez-Chantar, M. U. Latasa, J. M. Mato, and M. A. Avila. NO sensitizes rat hepatocytes to hepatocyte growth factor-induced proliferation through the modulation of S-adenosylmethionine levels. Gastroenterology 122:1355-1363, 2002.
22. Martínez-Chantar, M. L., M. Vázquez-Chantada, M. Garnacho-Echevarria, M. U. Latasa, M. Varela-Rey, J. Dotor, M. Santamaria, L. A. Martinez-Cruz, L. A. Parada, SC. Lu, and J. M. Mato. 5-Adenosylmethionine Regulates Cytoplasmic HuR Via AMP-Activated Kinase. Gastroenterology 131:223-232, 2006.
23. Wang, W., J. Fan, X. Yang, S. Furor, I. Lopez de Silanes, C. von Kobbe, J. Guo, S. N. Georas, F. Foufelle, D. G. Hardie, D. Carling, and M. Grospe. AMP-activated kinase regulates cytoplasmic HuR. Mol. Cell. Biol. 22:3425-3436, 2002.
24. Finkelstein, J. D. Methionine metabolism in mammals. J. Nutr. Biochem. 1:228-237, 1990.
25. Sullivan, D. M., and J. Hoffman. Fractionation and kinetic properties of rat liver and kidney methionine adenosyltransferase isozymes. Biochemistry 22:1636-1641, 1983.
26. Halim, A., L. LeGros, A. Geller, and M. Kotb. Expression and functional interaction of the catalytic and regulatory subunits of human methionine adenosyltransferase in mammalian cells. J. Biol. Chem. 274:29720-29725, 1999.
27. Mato, J. M., F. J. Corrales, S. C. Lu, and M. A. Avila. S-Adenosylmethionine: a control switch that regulates liver function. FASEB J. 16:15-26, 2002.
28. Avila, M. A., C. Berasain, L. Torres, A. Martin-Duce, F. J. Corrales, H. P. Yang, J. Prieto, S. C. Lu, J. Caballeria, J. Rodes, and J. M. Mato. Reduced mRNA abundance of the main enzymes involved in methionine metabolism in human liver cirrhosis and hepatocellular carcinoma. J. Hepatol. 33:907-914, 2000.
29. Lee, T. D., M. E. Sadda, M. H. Mendler, T. Bottiglieri, G. Kanel, J. M. Mato, and S. C. Lu. Abnormal hepatic methionine and GSH metabolism in patients with alcoholic hepatitis. Alcoholism: Clin. and Exp. Res. 28:173-181, 2004.
30. Huang, Z. Z., J. M. Mato, G. Kanel, and S. C. Lu. Differential effect of thioacetamide on hepatic methionine adenosyltransferase expression. Hepatology 29:1471-1468, 1999.
31. Lu, S. C., Z. Z. Huang, H. P. Yang, J. M. Mato, M. A. Avila, and H. Tsukamoto. Changes in methionine adenosyltransferase and S-adenosylmethionine homeostasis in the alcoholic rat liver. Am. J. Physiol. 279:G178-185, 2000.
32. Yang, H. P., Z. Z. Huang, Z. H. Zeng, C. J. Chen, J. H. Wang, and S. C. Lu. The role of c-Myb and Sp1 in the up-regulation of methionine adenosyltransferase 2A gene expression in human hepatocellular carcinoma. FASEB J. 15:1507-1516, 2001.
33. Yang, H. P., Z. Z. Huang, Z. H. Zeng, C. J. Chen, R. R. Selby, and S. C. Lu. Role of promoter methylation in increased methionine adenosyltransferase 2A expression in human liver cancer. Am. J. Physiol. 280:G184-190, 2001.
34. Yang, H. P., M. R. Sadda, V. Yu, Y. Zeng, T. D. Lee, X. P. Ou, L. X. Chen, and S. C. Lu, Induction of human methionine adenosyltransferase 2A expression by tumor necrosis factor alpha: Role of NF-κB and AP-1. J. Biol. Chem. 278:50887-50896, 2003.
35. Latasa, M. U., A. Boukaba, E. R. García-Trevijano, L. Torres, J. L. Rodríguez, J. Caballeria, S. C. Lu, G. López-Rodas, L. Franco, J. M. Mato, M. A. Avila. Hepatocyte growth factor induces MAT2A expression and histone acetylation in rat hepatocytes. Role in liver regeneration. FASEB J 10:1096, 2001.
36. Martínez-Chantar, M. L., E. R. Garcia-Trevijano, M. U. Latasa, A. Martin-Duce, P. Fortes, J. Caballeria, M. A. Avila, and J. M. Mato. Methionine Adenosyltransferase II β Subunit Gene Expression Provides a Proliferative Advantage in Human Hepatoma. Gastroenterology 124:940-948, 2003.
37. Tobeña R, Horikawa S, Calvo V, Alemany S. Interleukin-2 induces □-S-adenosyl-L-methionine synthetase gene expression during T-lymphocyte activation. Biochem. J. 1996; 319:929-933.
38. Garcia-Trevijano, E. R., M. U. Latasa, M. V. Carretero, C. Berasain, J. M. Mato, and M. A. Avila. S-Adenosylmethionine regulates MAT1A and MAT2A gene expression in cultured rat hepatocytes: a new role for S-adenosylmethionine in the maintenance of the differentiated status of the liver. FASEB J 14:2511-2518, 2000.
39. Martínez-Chantar, M. L., M. U. Latasa, M. Varela-Rey, S. C. Lu, E. García-Trevijano, J. M. Mato, M. A. Avila. L-Methionine availability regulates the expression of methionine adenosyltransferase 2A gene in human hepatocarcinoma cells. J Biol Chem 278:19885-19890, 2003.
40. Martínez-Chantar M L, Corrales F J, Martínez-Cruz A, García-Trevijano E R, Huang Z Z, Chen L X, Kanel G, Avila M A, Mato J M, Lu S C. Spontaenous oxidative stress and liver tumors in mice lacking methionine adenosyltransferase 1A. FASEB J 10:1096, 2002; June 7.
41. Santamaría, E., M. A. Avila, M. U. Latasa, A. Rubio, A. Martin-Duce, S. C. Lu, J. M. Mato, F. J. Corrales. Functional proteomics of non-alcoholic steatohepatitis: mitochondrial proteins as targets of 5-adenosylmethionine. Proc Nat Acad Sci USA 100:3065-3070, 2003.
42. LeGros Jr., H. L., A. M. Geller, and M. Kotb. Differential regulation of methionine adenosyltransferase in superantigen and mitogen stimulated human T lymphocytes. J. Biol. Chem. 272:16040-16047, 1997.
43. Moyer, M. P., L. A. Manzano, R. L. Merriman, J. S. Stauffer, and L. R. Tanzer. Letter to the Editor: NCM460, a normal human colon mucosal epithelial cell line. In vitro Cell Dev. Biol-Animal 32:315-317, 1996.
44. Mato, J. M., and S. C. Lu. S-adenosylmethionine. In: Encyclopedia of Dietary Supplements (Coates P M, Blackman M R, Levine M, Moss J, White J D, editors), Marcel Dekker, Inc., New York, USA (2005).
45. Ito, K., S. Ikeda, N. Kojima, M. Miura, K, Shimizu-Saito, I. Yamaguchi, I. Katsuyama, K. Sanada, T. Iwai, H. Senoo, and S. Horikawa. Correlation between the expression of methionine adenosyltransferase and the stages of human colorectal carcinoma. Surgery Today 30:706-710, 2000.
46. Sodir N M, Chen X, Park R, Nickel A E, Conti P S, Moats R, Bading J R, Shibata D, Laird P W. Smad3 deficiency promotes tumorigenesis in the distal colon of Apc^{Min/+} mice. Cancer Res 2006; 66:8430-8438.
47. Hardwick J C H, Van Den Brink G R, Offerhaus G J, Van Deventer S J H, Peppelenbosch M R Leptin is a growth factor for colonic epithelial cells. Gastroenterology 2001; 121:79-90.
48. Vandesomeple J, De Preter K, Pattyn F, Poppe B, Van Roy N, De Paepe A, Speleman F. Accurate normalization of real-time quantitative RT-PCR data by geometric averaging of multiple internal control genes. Genome Biology 2002; 3:34.1-34.11.
49. Giulietti A, Overbergh L, Valckx D, Decallonne B, Bouillon R, Mathieu C. An overview of real-time quantitative PCR: applications to quantify cytokine gene expression. Methods 2001; 25:386-401.
50. Lee T D, Yang H P, Whang J, Lu S C. Cloning and characterization of the human glutathione synthetase 5′-flanking region. Biochem. J. 2005; 390:521-528.
51. Farrar C, Clarke S. Altered levels of S-adenosylmethionine and S-adenosylhomocysteine in the brains of L-isoaspartyl (D-aspartyl) O-methyltransferase-deficient mice. J. Biol. Chem. 2002; 277:27856-27863.
52. Brown N D, Strickler M P. Femtomolar ion-pair high-performance liquid chromatographic method for determining Dns-polyamine derivatives of red blood cell extracts utilizing an automated polyamine analyzer. J. Chromatogr. 1982; 245:101-108.
53. Clarke S G. Inhibition of mammalian protein methyltransferases by 5′-methylthioadenosine (MTA): a mechanism of action of dietary SAMe? The Enzymes 2006; 24:467-493.
54. LeGros Jr., H. L., A. B. Halim, A. M. Geller, and M. Kotb. Cloning, expression, and functional characterization of the 13 regulatory subunit of human methionine adenosyltransferase (MAT II). J. Biol. Chem. 275:2359-2366, 2000.
55. LeGros, L., H. B. Halim, M. E. Chamberlin, A. Geller, and M. Kotb. Regulation of the human MAT2β gene encoding the regulatory 13 subunit of methionine adenosyltransferase, MAT II. J. Biol. Chem. 276:24918-24924, 2001.
56. Komninou, D., A. Ayonote, J. P. Richie, Jr., and B. Rigas. Insulin resistance and its contribution to colon carcinogenesis. Exp. Biol. Med. 228:396-405, 2003.
57. Rouet-Benzineb, P., T. Aparicio, S. Guilmeau, C. Pouzet, V. Descatoire, M. Buyse, and A. Bado. Leptin counteracts sodium butyrate-induced apoptosis in human colon cancer HT29 cells via NF-κB signaling. J. Biol. Chem. 279:16495-16502, 2004.
58. Barrenetxe, J., A. C. Villaro, L. Guembe, I. Pascual, M. Munoz-Navas, A. Barber, and M. P. Lostao. Distribution of the long leptin receptor isoform in brush border, basolateral membrane, and cytoplasm of enterocytes. Gut 50:797-802, 2002.
59. De La Rosa, J., H. L. LeGros Jr, A. M. Geller, and M. Kotb. Changes in the relative amount of subunits of methionine adenosyltransferase in human lymphocytes upon stimulation with a polyclonal T cell mitogen. J. Biol. Chem. 267:10699-10704, 1992.
60. Darmoul, D., V. Gratio, H. Devaud, and M. Laburthe. Protease-activated receptor 2 in colon cancer. J. Biol. Chem. 279:20927-20934, 2004.
61. Lin, J., P. W. Hsiao, T. H. Chiu, and J. I. Chao. Combination of cyclooxygenase-2 inhibitors and oxaliplatin increases the growth inhibition and death in human colon cancer cells. Biochem. Pharmacol. 70:658-667, 2005.
62. Nagai, H., K. Matsumaru, G. Feng, and N. Kaplowitz. Reduced glutathione depletion causes necrosis and sensitization to tumor necrosis factor-alpha-induced apoptosis in cultured mouse hepatocytes. Hepatology 36:55-64, 2002.
63. Nitta, T., K. Igarashi, and N. Yamamoto. Polyamine depletion induces apoptosis through mitochondria-mediated pathway. Exp. Cell Res. 276:120-128, 2002.
64. Gerner, E. W., and F. L. Meyskebs Jr, Polyamines and cancer: old molecules, new understanding. Nature Reviews Cancer 4:781-792, 2004.
65. Scorcioni, F., A. Corti, P. Davalli, S. Astancolle, and S. Bettuzzi. Manipulation of the expression of regulatory genes of polyamine metabolism results in specific alterations of the cell-cycle progression. Biochem J. 354:217-223, 2001.
Seiler, N., and F. Raul. Polyamines and apoptosis. J. Cell Mol. Med. 9:623-642, 2005.
66. Thomas, T., and T. J. Thomas. Polyamines in cell growth and cell death: molecular mechanisms and therapeutic applications. Cell Mol Life Sci. 58:244-258, 2001.
67. Yang, H. P., M. R. Sadda, M. Li, Y. Zeng, L. X. Chen, W. J. Bae, X. P. Ou, M. T. Runnegar, J. M. Mato, and S. C. Lu. S-Adenosylmethionine and its metabolite induce apoptosis in HepG2 cells: role of protein phosphatase 1 and Bcl-xS. Hepatology 40:221-231, 2004.
68. Ferris, H. A., R. E. Carroll, M. M. Rasenick, and R. V. Benya. Constitutive activation of the gastrin-releasing peptide receptor expressed by the nonmalignant human colon epithelial cell line NCM460. J. Clin. Invest. 100:2530-2537, 1997.
69. Sahi, J., S. G. Nataraja, T. L. Layden. J. L. Goldstein, M. P. Moyer, and M. C. Rao. Cl-transport in an immortalized human epithelial cell line (NCM460) derived from the normal transverse colon. Am. J. Physiol. 275:C1048-1057, 1998.
70. Said, H. M., A. Ortiz, V. S. Subramanian, E. J. Neufeld, M. P. Moyer, and P. K. Dudeja. Mechanism of thiamine uptake by human colonocytes: studies with cultured colonic epithelial cell linen NCM460. Am. J. Physiol. 281:G144-150, 2001.
71. Avila, M. A., E. R. García-Trevijano, S.C. Lu, F. J. Corrales, and J. M. Mato. Molecule in focus: Methylthioadenosine. Int. J. Biochem Cell Biol. 36:2125-2130, 2004.
72. Sharp, D. A., D. A. Lawrence, and A. Ashkenazi. Selective knockdown of the long variant of cellular FLICE inhibitory protein augments death receptor-mediated caspase-8 activation and apoptosis. J. Biol. Chem. 280:19401-19409, 2005.
73. Thorburn, A. Death receptor-induced cell killing. Cell Signalling 16:139-144, 2004.
74. Zhang, X., T. G. Jin, H. Yang, W. C. DeWolf, R. Khosravi-Far, and A. F. Olumi. Persistent c-FLIP(L) expression is necessary and sufficient to maintain resistance to tumor necrosis factor-related apoptosis-inducing ligand-mediated apoptosis in prostate cancer. Cancer Res 64:7086-7091, 2004.
75. Benoit, V., A. Chariot, L. Delacroix, V. Deregowski, N. Jacobs, M. P. Merville; and V. Bours. Caspase-8-dependent HER-2 cleavage in response to tumor necrosis factor alpha stimulation is counteracted by nuclear factor KB through c-FLIP-L expression. Cancer Res 64:2684-2691, 2004.
76. Galligan, L., D. B. Longley, M. McEwan, T. R. Wilson, K. McLaughlin, and P. G. Johnston. Chemotherapy and TRAIL-medicated colon cancer cell death: the roles of p53, TRAIL receptors, and c-FLIP. Mol Cancer Ther 4:2026-2036, 2005.
77. Zhou, X. D., J. P. Yu, H. X. Chen, H. G. Yu, and H. S. Luo. Expression of cellular FLICE-inhibitory protein and its association with p53 mutation in colon cancer. World J Gastro 11:2482-2485, 2005.
78. Longley, D. B., T. R. Wilson, M. McEwan, W. L. Allen U. McDermott, L. Galligan, and P. G. Johnston. C-FLIP inhibits chemotherapy-induced colorectal cancer cell death. Oncogene 25:838-848, 2006.
79. Laird, P. W., L. Jackson-Grusby, A. Fazeli, S. L. Dickinson, W. E. Jung, E. Li, R. A. Weinberg, and R. Jaenisch. Suppression of intestinal neoplasia by DNA hypomethylation. Cell 81:197-205, 1996.
80. Boivin, G. P., K. Washington, K. Yang, J. M. Ward, T. P. Pretlow, R. Russell, D. G. Besselsen, V. L. Godfrey, T. Doetschman, W. F. Dove, H. C. Pitot, R. B. Halberg, S. H. Itzkowitz, J. Groden, and R. J. Coffey. Pathology of mouse models of intestinal cancer: consensus report and recommendations. Gastroenterology 124:762-777, 2003.
81. Paulsen, J. E., E. Namork, I. L. Steffensen, T. J. Eide, and J. Alexander. Identification and quantification of aberrant crypt foci in the colon of Min mice—a murine model of familial adenomatous polyposis. Scand. J. Gastro. 35:534-539, 2000.
82. Paulsen, J. E., I. L. Steffensen, E M Lçberg, T. Husoy, E. Namork, and J. Alexander. Qualitative and quantitative relationship betwseenm dysplastic aberrant erupt foci and tumrigenesis in the Min/+mouse colon.

Cancer Res. 61:5010-5015, 2001.
83. Kim Y I Role of folate in colon cancer development and progression. J. Nutr. 2003; 133:37315-37395.
84. Giovannucci E, Rimm EB, Ascherio A, Stampfer M J, Colditz G A, Willett W C. Alcohol, low-methionine-low-folate diets, and risk of colon cancer in men. J. Natl. Cancer Inst. 1995; 87:265-273.
85. Laird P W, Jackson-Grusby L, Fazeli A, Dickinson S L, Jung WE, Li E, Weinberg R A, Jaenisch R. Suppression of intestinal neoplasia by DNA hypomethylation. Cell 1995; 81:197-205.
86. Dante R, Anaud M, Niveleau A. Effects of 5′deoxy-5′-methylthioadenosine on the metabolism of S-adenosylmethionine. Biochem Biophys Res Commun 1983; 114:214-221.
87. Latasa M U, Boukaba A, GarcÌa-Trevijano ER, Torres L, Rodríguez J, Caballería L, Lu S C, López-Rodas G, Franco L, Mato J M, Avila M A. Hepatocyte growth factor induces MAT2A expression and histone acetylation in rat hepatocytes. Role in liver regeneration. FASEB J 2001; March 5.10.10961.00-0556e.
88. Veal N, Hsieh C L, Xiong S, Mato J M, Lu S C, Tsukamoto H. Inhibition of lipopolysaccharide-stimulated TNF□ promoter activity by S-adenosylmethionine and 5′-methylthioadenosine. Am. J. Physiol. 2004; 287: G352-362.
89. Feinberg AP, Tycko B. The history of cancer epigenetics. Nat Rev Cancer 2004; 4:143-143.
90. Mato, J. M., L. Alvarez, P. Ortiz, and M. A. Pajares. S-adenosylmethionine synthesis: molecular mechanisms and clinical implications. Pharmacol Ther 73:265-280, 1997.
91. Thomas, T., and T. J. Thomas. Polyamines in cell growth and cell death: molecular mechanisms and therapeutic applications. Cell Mol Life Sci. 58:244-258, 2001.
92. Bashir, O., A. J. Fitzgerald, J. Berlanga-Acosta, R. J. Playford, and R. A. Goodlad. Effect of epidermal growth factor administration on intestinal cell proliferation, crypt fission and polyp formation in multiple intestinal neoplasia (Min) mice. Clinical Sci 105:323-330, 2003.
93. Chen, L., Y. Zeng, H. P. Yang, T. D. Lee, S. W. French, F. J. Corrales, E. R. García-Trevijano, M. A. Avila, J. M. Mato, and S. C. Lu. Impaired liver regeneration in mice lacking methionine adenosyltransferase 1A. FASEB J 10.1096/fj.03-1204e, March 2004.

1-18. (canceled)

19. A pharmaceutical composition for use in treating cancer, comprising:
at least one of SAMe or MTA; and
a pharmaceutically suitable carrier.

20. The composition of claim 19, wherein said cancer is an epithelial cancer.

21. The composition of claim 19, wherein said cancer is one selected from breast, prostate, colorectal, lung, colon, bladder, head and neck, intestine, ovarian, or skin cancer, or a combination thereof.

22. The composition of claim 19, wherein said cancer is a mammalian cancer.

23. The composition of claim 19, further comprising an apoptosis inducing agent.

24. The composition of claim 23, wherein said apoptosis inducing agent is a TNF-related apoptosis-inducing ligand.

25. A pharmaceutical composition for use in chemoprevention of cancer, comprising:
at least one of SAMe or MTA; and
a pharmaceutically suitable carrier.

26. The composition of claim 25, wherein said cancer is an epithelial cancer.

27. The composition of claim 25, wherein said cancer is one selected from breast, prostate, colorectal, lung, colon, bladder, head and neck, intestine, ovarian; or skin cancer, or a combination thereof.

28. The composition of claim 25, wherein said cancer is a mammalian cancer.

29. A composition for use in inducing apoptosis in cancerous cells, comprising:
at least one of SAMe or MTA.

30. The composition of claim 29, wherein said cancerous cells are epithelial cells.

31. The composition of claim 29, wherein said cancerous cells are selected from breast, prostate, colorectal, lung, colon, bladder, head and neck, intestine, ovarian, or skin cancer cell, or a combination thereof.

32. A pharmaceutical composition for use as an adjunct to a chemotherapeutic agent, comprising:
at least one of SAMe or MTA; and
a pharmaceutically suitable carrier.

33. The composition of claim 32, wherein said chemotherapeutic agent is a TNF-related apoptosis inducing agent.

34. A composition for use in blocking the expression of MAT2A and/or c-FLIP in cells, comprising:
at least one of SAMe or MTA.

35. A method of treating cancer in a subject, comprising the step of:
administering to the subject a pharmaceutical composition at a predetermined dosage and duration,
wherein said composition comprises at least one of SAMe or MTA.

36. The method of claim 35, wherein said administering step is one selected from parenteral, oral, subcutaneous, transdermal, transmucosal, intramuscular, or rectal administration.

37. The method of claim 35, wherein step administering step is performed via intravenous infusion, intramuscular infusion, or enema injection.

38. The method of claim 35, wherein said composition is administered to the subject at a dosage of at least 150 mg/kg/d of SAMe or at least 75 mg/kg/d of MTA or both.

39. The method of 35, wherein said composition further comprising a chemotherapeutic agent.

40. The method of claim 39, wherein said chemotherapeutic agent is a TNF-related apoptosis inducing agent.

41. The method of claim 35, wherein said subject is one suffering from an epithelial cancer.

42. The method of claim 35, wherein said subject is one suffering from breast, prostate, colorectal, lung, colon, bladder, head and neck, intestine, ovarian, or skin cancer cell, or a combination thereof.

43. The method of claim 35, wherein said duration is at least 16 hours.

44. A chemoprevention method for an epithelial cancer, comprising:
administering a pharmaceutical composition to a subject at a predetermined dosage and duration,
wherein said pharmaceutical composition comprises at least one of SAMe or MTA.

45. The method of claim 44, wherein said predetermined dosage is about 5 mM for SAMe and about 2 mM MTA, or at least 75 mg/kg/day for both SAMe and MTA.

46. The method of claim 44, wherein said subject is a remitted cancer patient.

47. The method of claim 44, wherein said cancer is one selected from breast, prostate, colorectal, lung, colon, bladder, head and neck, intestine, ovarian, or skin cancer cell, or a combination thereof.

48. A method for selectively inducing apoptosis in a population of epithelial cells, comprising:
contacting said population of epithelial cells with at least one of SAMe or MTA at a concentration of about 0.5 mM or higher for a duration of 16 hours or more,
wherein said population of epithelial cells contain at least one cancerous cell, and whereby contacting said population of cells with SAMe or MTA or both results selective induction of apoptosis in the cancerous cell.

49. The method of claim 48, wherein said epithelial cells comprises one selected from breast, prostate, colorectal, lung, colon, bladder, head and neck, intestine, ovarian, or skin cancer cell, or a combination thereof.

50. A method of diagnosing cancer in a subject, comprising:
detecting expression of MAT2A and MAT2β in a sample of epithelial cells obtained from a subject; and
comparing the expression level of MAT2A and MAT2β in the sample to a normal reference,
whereupon if the expression level of MAT2A and MAT2β in the sample is higher than the normal reference, a diagnosis of cancer is indicated.

51. The method of claim 50, wherein said epithelial cells are selected from breast, prostate, colorectal, lung, colon, bladder, head and neck, intestine, ovarian, or skin cell, or a combination thereof.

52. A method of cancer diagnosis, comprising the steps of:
detecting expression of MAT in a sample of epithelial cells obtained from a subject; and
comparing the expression level in the sample to a normal reference,
wherein if an abnormal level of MAT expression is detected in the sample, a cancer diagnosis is indicated.

53. The method of claim 52, wherein said epithelial cells are selected from breast, prostate, colorectal, lung, colon, bladder, head and neck, intestine, ovarian, or skin cell, or a combination thereof.

54. A systematic method of developing a pharmaceutical composition for use in treating cancer, comprising:
selecting an apoptosis-inducing agent known to have cancer cells resistant to it; and
applying said apoptosis-inducing chemotherapeutic agent and at least one of SAMe or MTA to said resistant cancer cells;
whereby a pharmaceutical composition is developed if the combination is more effective in inducing apoptosis in the cancer cells than the chemotherapeutic agent alone.

55. The method of claim 54, wherein said apoptosis-inducing agent is a TNF-related apoptosis-inducing ligand.
